(19) United States
(12) Patent Application Publication
(10) Pub. No.: US 2006/0029615 A1
(43) Pub. Date: Feb. 9, 2006

(54) Novel recombinant T4 phage particle and uses thereof

(76) Inventors: Zhao-jun Ren, Monmouth Junction, NJ (US); Darhsing Lai, Yardley, PA (US)

Correspondence Address: CHERYL H AGRIS PHD, PO BOX 806, PELHAM, NY, 10803, US

(21) Appl. No.: 11/188,236

(22) Filed: Jul. 22, 2005

(30) Foreign Application Priority Data
Aug. 6, 2004 (CN) 200410040389.9

(51) Int. Cl. A61K 39/02; C12N 7/00; C12N 15/74

(52) U.S. Cl. 424/200.1; 435/472; 435/235.1

(57) ABSTRACT

The invention is directed to a novel recombinant T4 phage particle expressing a HOC and/or SOC fusion peptide as well as methods for their preparation and methods of use in compositions and kits.

### PRIORITY CLAIM

This application claims priority to Chinese application no. 200410040389.9, filed Aug. 6, 2004 under 37 U.S.C. § 119(a)-(d), the contents of which are incorporated herein by reference.

### FIELD OF THE INVENTION

The invention is directed to a novel recombinant T4 phage particle expressing a HOC and/or SOC fusion peptide or protein as well as methods for their preparation and methods of use in compositions and kits.

### BACKGROUND OF THE INVENTION

A number of attempts have been made to express heterologous proteins in bacteriophages (reviewed in Adhya et al., 2005, Mol. Microbiol. 55: 1300-14). Filamentous bacteriophages M13 and fd have subsequently been extensively used to display proteins and short peptides on the minor capsid protein pII (see, for example, Devlin et al., 1990, Science 249:404-406; Parmley and Smith., 1998, Gene 73:305-318; Perham et al., 1995, FEMS Microbiol. Rev. 17: 25-31; U.S. Pat. No. 6,420,113, U.S. Pat. No. 6,555,310, U.S. Pat. No. 6,057,098, Smith, 1985, Science 228:1315-1317) and major capsid protein pVIII (Greenwood et al., 1991, J. Mol. Biol. 220:821-827; Kang et al., 1991, Proc. Natl. Acad. Sci. USA 88:4363-4366). Icosahedral phage λ has also been used to display foreign proteins on the outer capsid protein gpD (Mikawa et al., 1996, J. Mol. Biol. 262: 21-30; Sternberg and Hoess, 1995, Proc. Natl. Acad. Sci. USA 92: 1609-1613) and on tail protein gpV (Dunn, 1995, J. Mol. Biol. 248:497-506; Maruyama et al., 1994, Proc. Natl. Acad. Sci. USA 91:8273-8277). Phage T7 can also display proteins on the capsid (O'Neil and Hoess, 1995, Curr. Opin. Struc. Biol. 5(4): 443-449; WO98/05344; U.S. Pat. No. 6,777,239 and Maruyama et al., 1994, Proc. Natl. Acad. Sci. USA 91:8273-8277).

However, there are significant limitations. For example, display of certain peptides is restricted when filamentous phage is used, or not possible, since the fused peptide has to be secreted through the E. coli membranes as part of the phage assembly apparatus. Since both pill and pVIII proteins are essential for phage assembly, it is difficult to display large domains or full-length proteins without interfering with their essential biological functions. In situations where large peptide sequences are displayed, their copy number per phage capsid is greatly reduced and unpredictable. Similar problems on the size and copy number have been encountered with the phage lambda display systems. It is often necessary to incorporate wild type protein molecules along with the recombinants to generate viable phage using either a helper phage or a partial genetic suppression of amber mutant (Hoess, 2002, Curr. Pharm. Biotechnol. 3:23-8).

T4 Expression Systems

The phage T4 capsid contains three essential structural proteins: major capsid protein gp23 (930 copies per capsid), and two minor capsid proteins: gp24 (vertex protein, 55 copies) and gp20 (portal vertex protein 12 copies) (Black et al., 1994, Morphogenesis of the T4 head, p. 218-253. In J. D. Karam (ed), Bacteriophage T4.ASM Press, Washington, D.C.; Yanagide, 1997, J. Mol. Biol. 109:515-537; Fokine et al., 2004, Proc. Natl. Acad. Sci. (USA) 101, 6003-6008). In addition, the T4 virion outer surface is coated with two dispensable capsid proteins SOC (810 copies per capsid, molecular mass 9 kDa) and HOC (155 copies, 40 kDa). These proteins are regularly displayed on the T4 icosahedral lattice (Black et al., 1994, Morphogenesis of the T4 head, p. 218-253. In J. D. Karam (ed), Bacteriophage T4.ASM Press, Washington, D.C.; Yanagide, 1997, J. Mol. Biol. 109:515-537). SOC and HOC bind strongly to the capsid following capsid assembly and the capsid expansion triggered by DNA packaging that creates HOC and SOC capsid binding sites (Jiang et al., 1997, Infect. Immun. 65: 4770˜4777).

The use of T4 phage as a vector to express heterologous peptides or proteins has been explored (for early work see Casna and Shub, 1982, Gene 18:297-307). A variety of approaches have been attempted. Shub and Casna, 1985, Gene 37: 31-36, discloses the expression of an rIIB-lacZ gene fusion in T4 phage. JP62232384 discloses a dC-type recombinant T4 phage containing heterologous DNA as well as a method of expression in E. coli by simultaneous infection with the recombinant T4 phage and T4 phage having a normal gammall gene. Singer and Gold, 1991, Gene 106:1-6, discloses a T4 expression system that contains the multiple cloning site of pUC18/19 and T7 promoter and terminator. Hong et al., 1993, Gene 136:193-198 and Hong and Black, 1993, Virol. 194:481-490, discloses a T4 packaging system using the T4 non-essential capsid scaffold protein IPIII. Asimov et al., 1995, Virus Genes 10: 173-177, discloses construction of Homeric T4 displaying foreign peptides. WO0/06717 discloses a method for improving efficiency in phage display by modifying the coat protein.

Rao et al., 1992, Gene 113:25-33, discloses the use of a T4 packaging system to package limm434 DNA into proheads. Rao and Leffers, 1993, Virology 196:896-899 further discloses the construction of empty proheads and the use of these proheads as tools for expressing heterologous DNA.

Mullaney and Black, 1998, BioTechniques 25:1008-1012 discloses a T4 phage derived protein expression, packaging and processing system where an HIV-1 protease is fused to green fluorescent protein. The fusion protein is targeted within the phage with an N-terminal capsid targeting sequence.

Another approach has involved the display of heterologous proteins on T4 by their fusion to capsid proteins SOC or HOC. Jiang et al., 1997, Infection and Immunity 65:4770-4777, discloses the cloning of a 36 amino acid PorA peptide from Neisseria meningitides into T4 display vectors to generate fusions at the N terminus of HOC or SOC.

Ren et al., 1996, Protein Science 5:1833-1843 discloses C-terminal fusions of a tetrapeptide, the 43 residue V3 loop domain of HIV gp120, and poliovirus VP1 capsid protein (312 residues) to SOC. Ren et al., 1997, Gene 198:303-311 discloses construction of a T4 phage hoc gene display vector.

Ren and Black, 1998, Gene 215:439-444, discloses the display of full-length heterologous protein by fusion to SOC or HOC. The vectors contained either SOC or HOC. This article discloses the display of a 271 residue heavy and light chain fused IgG anti-egg while lysozyme to the COOH-terminus of the SOC capsid protein and HIV-1 CD4 receptor (183 amino acids) fused with the amino-terminus of HOC. Malys et al., 2002, J. Mol. Biol. 319:289-304, discloses a bipartite T4 phage display library containing SOC and HOC randomized peptide fusions displayed on the external capsid surface of T4. Specifically, SOC and HOC DNAs are attached to random five amino acid sequence peptide extensions to the C terminus of SOC via a tetra-alanine linker. The recombinant particle disclosed in both of these articles did not contain T4 IPII and IPIII proteins; it only contained the IPI and was e⁺, denV⁺ and alt⁺.

### SUMMARY OF THE INVENTION

The invention is directed to a recombinant T4 phage particle having or expressing one or more heterologous peptides, nucleic acids or genes, particularly a T4 HOC and/or SOC fusion peptide (also includes proteins). The term “peptide” as used herein encompasses peptide, polypeptide and protein sequences containing 4 or more amino acids and includes full length proteins.

The HOC or SOC protein or functional portion thereof is bound to a heterologous peptide. The heterologous peptide may, in a particular embodiment, contain up to about 1,150 amino acids and may encompass one or more proteins, HOC or SOC sequence. The heterologous peptide may be separated by a spacer or linker. In particular, the recombinant T4 phage particle contains or expresses a heterologous peptide such as a receptor, ligand, antigen, immunogen or toxin and antibody (e.g., IgG-Fab/Fv single or double chain). Examples of such heterologous peptides (including proteins) include, but are not limited to, a cytokine, CD4 receptor, vascular endothelial growth factor receptor (VEGFR), vascular endothelial growth factor (VEGF), epidermal growth factor (EGF), epidermal growth factor receptor (EGFR), Bacillus thuringiensis toxins (Cry or Cyt gene expression products), eV_{H}-link-V_{L}. The antigen or immunogen may be derived from a pathogen. As defined herein, “derived from” means that the antigen or immunogen contains peptide sequences from the particular pathogen. The pathogen may be a parasite, insect, bacteria, fungus or virus which may have a deleterious effect on an animal or plant. The peptides may be derived from Human Immunodeficiency Virus (HIV) (e.g., gp120 V3 loop, p24m gp41, tat, and/or vpr), Foot and Mouth Disease Virus (FMDV) (e.g., VP1, VP2, VP3 and/or VP4), poliovirus (e.g., poliovirus VP1), avian encephalomyelitis (AEV), Avian Influenza Virus (AIV) (e.g., HA protein), avian adenovirus, avian leukosis virus (ALV), avian virus, Fowipox virus (FPV), human Dengue Fever Virus, infectious bursal disease virus (IBDV), Marek's disease virus (MDV), hepatitis B virus (e.g., Hepatitis B viral capsid antigen (HBcAg), Hepatitis B viral envelope protein (HBeAg), human measles virus, encephalitis (VP1) and classical swine fever virus (CSFV) (e.g., E2, mE2). In another embodiment, the pathogen is a bacteria, particularly a mycobacterium (tuberculosis, e.g., purified protein derivative (PPD), Bacillus of Calmette and Guerin (BCG) antigen and/or A60-antigen), Streptococcus iniae, Streptocococcus agalactiae, Flavobacterium columnare, Edwardsiella tarda, Vibrio anguillarum biotype I and II, V. Harveyi, V. parahaemolyticus and V. vulnificus, Bacillus thuringiensis (Cry or Cyt). In a related aspect, the invention is directed to a recombinant prokaryotic cell comprising the T4 phage particle of the present invention.

In a particular embodiment, the invention is directed to A recombinant T4 phage particle comprising a SOC and/or HOC fusion peptide, wherein said phage particle has inactive T4 endonuclease V and/or T4 lysozyme function and wherein said fusion peptide comprises SOC and/or HOC bound to one or more heterologous peptides selected from the group consisting of a a tumor antigen or receptor peptide selected from the group consisting of epidermal growth factor (EGF), epidermal growth factor receptor (EGFR), vascular endothelial growth factor (VEGF) and vascular endothelial growth factor receptor (VEGFR); a toxin, antigen, immunogen derived from or antibody or fragments therof to a pathogen selected from the group consisting of HIV peptides selected from the group consisting of p24, gp41, tat and/or vpr antigenic or immunogenic peptides; FMDV Serotype O VPI, VPII, VPIII and/or VPIV protein; FMDV VPI, VPII, VPIII and/or VPIV from two or more serotypes; CFSV mE2 protein; CFSV mE2 and CFSV E2; avian adenovirus, avian leukosis virus (ALV), avian reovirus, Fowlpox virus (FPV), human Dengue Fever Virus, infectious bursal disease virus (IBDV), Marek's disease virus (MDV), hepatitis B virus, human measles virus, and classical swine fever virus (CSFV), canine distemper virus, feline distemper virus, canine adenovirus, mycobacterium, Streptococcus iniae, Streptocococcus agalactiae, Flavobacterium columnare, Edwardsiella tarda, Vibrio anguillarum biotype I and II, V. Harveyi, V. parahaemolyticus V. vulnificus and Bacillus thuringiensis (Cry or Cyt).

In a more particular embodiment, the invention is directed to a recombinant T4 phage particle expressing one or more SOC and/or HOC-FMDV fusion peptides, particularly a VP1 (1D), VP2 (1B), VP3 (1C) and/or VP4 (1A) protein or peptide having VP1, VP2, VP3 and/or VP4 function or alternatively expressing the P1 gene The FMDV peptide may be derived from FMDV serotype 0, FMDV serotype A, FMDV serotype C, FMDV serotype Asia-I, FMDV serotype Sat-1, FMDV serotype Sat-2 and FMDV serotype Sat-3.

In a related aspect, the invention is directed to an isolated fusion peptide comprising HOC or SOC bound to a heterologous peptide derived from a pathogen selected from the group consisting of Human Immunodeficiency Virus (HIV), Foot and Mouth Disease Virus (FMDV), poliovirus, avian encephalomyelitis (AEV), Avian Influenza Virus (AIV), avian adenovirus, avian leukosis virus (ALV), avian reovirus, Fowlpox virus (FPV), human Dengue Fever Virus, infectious bursal disease virus (IBDV), Marek's disease virus (MDV), hepatitis B virus, human measles virus, and classical swine fever virus (CSFV), mycobacterium, Streptococcus iniae, Streptocococcus agalactiae, Flavobacterium columnare, Edwardsiella tarda, Vibrio anguillarum biotype I and II, V. Harveyi, V. parahaemolyticus V. vulnificus, and Bacillus thuringiensis and a tumor antigen or receptor peptide selected from the group consisting of epidermal growth factor (EGF), epidermal growth factor receptor (EGFR), vascular endothelial growth factor (VEGF) and vascular endothelial growth factor receptor (VEGFR). These fusion peptides may be obtained from the T4 particle of the present invention. The invention further relates to a nucleic acid molecule encoding said fusion peptide as well as a nucleic acid construct, vector and prokaryotic cell comprising said nucleic acid molecule. In another related aspect, the nucleic acid molecule encoding the fusion peptide may comprise sequences encoding T4 IPII and IPIII or functional peptides thereof, but devoid of nucleic acid sequences encoding functional T4 lysozyme and/or endonuclease V.

In another embodiment, the recombinant T4 phage particle expresses a HOC and/or SOC fusion peptide and has inactive T4 endonuclease V and T4 lysozyme function which may alternatively be referred to as denV⁻ and e⁻ respectively. The recombinant T4 phage particle may further be ipII⁺ and ipIII⁺ or alternatively referred to as containing nucleic acid sequences encoding functional IPII and IPIII proteins.

The invention is further directed to methods for obtaining the recombinant phage particles of the present invention. In one embodiment, the method comprises (a) providing a vector comprising a nucleic acid sequence encoding HOC and/or SOC fusion peptide; (b) providing a recombinant hoc and/or soc negative expression system; (c) culturing a prokaryotic cell (e.g., E. coli) with the vector of (a) and expression system of (b) under conditions to obtain recombination of (a) and (b) and obtain said recombinant T4 particle and (d) isolating said recombinant T4 phage particle. In a related aspect, the invention is directed to a prokaryotic cell comprising said recombinant T4 phage particle.

In a related aspect, the invention is directed to a method for obtaining a HOC-fusion peptide (includes full length protein) by culturing a prokaryotic cell comprising said T4 phage particle under conditions suitable for the production of the peptide and isolating the peptide. In a further embodiment, the SOC and/or HOC-fusion peptide may contain a cleavage site. Therefore, the invention may further be directed to a method for obtaining a heterologous peptide comprising culturing a prokaryotic cell comprising the recombinant T4 phage of the present invention, isolating the SOC and/or HOC fusion heterologous peptide and cleaving the SOC and/or HOC sequence from the fusion heterologous peptide and isolating the heterologous peptide.

The invention is further directed to a hoc and/or soc negative (also referred to a hoc⁻ and/or soc⁻), and optionally ipII⁻ and ipIII⁻ T4 phage expression system. As defined herein, a T4 phage expression system is a system containing T4 expression control sequences. In a particular embodiment, the T4 phage expression system is e⁻, and/or denV⁻. The invention is further directed to a method for obtaining the T4 expression system of the present invention which is hoc or soc negative, comprising the steps of: (a) providing a hoc⁻ or soc⁻ T4 phage; (b) providing a hoc⁻ or soc⁻, e⁻, and/or denV⁻ T4 phage particle, wherein (a) and (b) are both hoc⁻ and/or both soc⁻; (c) culturing a prokaryotic cell with (a) and (b) under conditions promoting recombination of (a) and (b) and (d) isolating said expression system. A hoc⁻ and soc⁻ negative expression system may be obtained by (a) providing a hoc⁻ recombinant T4 phage expression system; (b) providing a soc⁻ recombinant T4 phage expression system and (c) culturing (a) and (b) in a prokaryotic cell under conditions promoting recombination of (a) and (b) and (d) isolating said expression system.

The invention is further directed to a composition comprising one or more of the T4 phage particles and/or HOC and/or SOC-fusion peptides of the present invention. These compositions may be antigenic or immunogenic compositions and may be used as vaccines. The compositions may comprise a plurality of recombinant T4 phage particles of the present invention, each comprising a different HOC or SOC fusion peptide. In a specific embodiment, the composition is multivalent vaccine and comprises T4 particles expressing immunogenic or antigenic peptides derived from two or more FMDV serotypes or two or more HIV peptides. Thus the invention is directed to a method for modulating the growth of a pathogen in an animal in need thereof comprising administering to said animal the composition of the present invention in an amount effective to modulate growth of a pathogen in said animal. The animal may be a mammal, such as dog, cat, human, cow, pig, bird, fish or shrimp. The invention is further directed to the use of the particles of the present invention for the manufacture of a medicament for modulating the growth of a pathogen in an animal.

In yet another embodiment, the invention is directed to a library of the recombinant T4 phage particles and/or HOC and/or SOC fusion peptides of the present invention. As defined herein, a “library” is a mixture of two or more recombinant T4 phage particles or HOC or SOC fusion peptides. The recombinant T4 phage particle may express a ligand, receptor, antibody, antigen and/or immunogen. The T4 particle may further contain a detectable label (e.g., colloid golden, green, red or yellow fluorescent protein, Biotin). A variety of libraries containing the recombinant T4 phage particles of the present invention may be constructed

1) Peptide library, in which the insert is a randomized amino acid (aa) covering all 20 amino acids where the peptides function as ligand, antigen, immunogen.

2) Antibody library. Antibody IgG-Fab/Fv single or double chain domain may be cloned (especially for monoantibody gene engineering library) into the expression system of the present invention where the scFV or dcFV peptide is on T4 phage surface.

These libraries, in one embodiment may be used to detect the presence or absence of a pathogen in a sample comprising incubating the library with said sample and detecting the presence or absence binding of a phage particle in said library to said sample, wherein binding of said a phage particle to said sample indicates the presence of said pathogen. If the library is used for diagnosis kit preparation, pathogen elicited antibody in blood, tissue is detected or alternatively, the pathogen itself may be detected.

These libraries may be used to identify ligands that bind to particular receptors and comprises the following steps: (a) providing the library of the present invention and (b) providing a recombinant T4 phage particle expressing a HOC or SOC fusion heterologous peptide, wherein said heterologous peptide is a receptor (c) incubating (a) and (b); (d) detecting binding of (a) and (b).

The T4 phage particles of the present invention may further be used as a pesticide by expressing one or Bacillus thuringiensis toxins including but not limited to Cry or Cyt genes. Therefore, the invention is directed to a method for modulating pathogen (e.g., insect, fungus) infestation in a plant in need thereof comprising applying to said plant an amount of the T4 phage particles of the present invention expressing a HOC and/or SOC fusion Bacillus thuringiensis peptide and/or HOC and/or SOC-fusion Bacillus thuringiensis peptide effective to modulate pathogen infestation. Similarly the invention is directed to the use of the T4 phage particles of the present invention expressing a HOC and/or SOC fusion Bacillus thuringiensis peptide and/or HOC and/or SOC-fusion Bacillus thuringiensis peptide for the manufacture of a medicament or pesticide for use in modulating pathogen infestation in a plant.

### BRIEF DESCRIPTION OF THE FIGURES

FIG. 1 shows the construction of ΦT4ΔSoc (T4 phage Soc expression vector).

FIG. 2A shows the the pHOC-mutant vector and FIG. 2B shows the construction of ΦT4ΔHoc (T4 phage HOC expression vector).

FIG. 3 shows ΦT4ΔSoc-ΔHoc (T4 phage Soc-Hoc bipartite expression vector).

FIG. 4A shows a p IN-Soc integration vector, 4B shows a p IN-Hoc integration vector.

FIG. 5 shows the strategy for constructing recombinant display phage T4-SOC/mE2 and T4-SOC/E2 from integration plasmid and phage vector T4-Zh⁻.

FIG. 6 shows expression of SOC/E2 fusion protein on 12% SDS-PAGE gel (Coomassie blue staining). Lanes: 1. host E. coli BL21 (DE3) as negative control; 2. pSE2 expressed SOC/E2 fused protein (53 kDa); 3. pSmE2 expressed SOC/mE2 fused protein (26 kDa); 4. Protein Marker as kDa.

FIG. 7 shows the antibody affinity of plasmid pSmE2 expressed SOC-mE2 fusion protein measured by ELISA. The antibodies were: MS, Mouse sera against mE2; MDNA, mouse sera against CSFV-E2 DNA vaccine; RS, rabbit sera against mE2; PS, pig sera against CSFV vaccine. +: positive serum antibody; −: non-immunized same animal sera as negative controls.

FIG. 8 shows plasmid pHOCE2 expressed fusion protein running on 12% PAGE. In FIG. 8A. The gel was stained with Coomassie blue: lane 1. over-expressed fusion antigen HOC/E2 of Mw 84 kDa; lane 2. protein markers 97, 66, 43 kDa; lane 3. host E. coli BL21(DE3) as negative control. FIG. 8B shows Western blotting assay of plasmid expression extract with pig antiserum against CSFV vaccine.

FIG. 9 shows PCR detection of the recombinant immunogen displaying T4 phages. Lane 1, 2, Phage T4-SOC/mE2 DNA as template with the 621 bp target band; 3: Phage vector T4-Zh⁻ DNA as negative control; 4, 5, Phage T4-SOC/E2 DNA as template with the 1365 bp band; 6, DNA bp markers (DL2000).

FIG. 10A shows protein bands of T4 phage display strain T4-SOC/mE2 on Coomassie blue staining of 12% SDS-PAGE. Lane 1. phage T4-SOC/mE2; the arrow indicates the 26 kDa SOC-mE2 band; 2. phage vector T4-Zh⁻. FIG. 10B Western blotting of FIG. 10A with pig CSFV-antiserum and rabbit anti-pig IgG conjugated with HRP.

FIG. 11 shows Dot-ELISA immune assay of recombinant phage T4 with pig anti-CSFV serum as first antibody, and rabbit anti-pig IgG conjugated with HRP as second antibody. The spots are: 1. phage T4-SOC/mE2—HOC/E2 dual sites display; 2. phage T4-SOC/mE2 single site display; 3. plasmid protein CSFV mE2 as positive control; 4. phage vector T4-Zh⁻.

FIG. 12 shows 12% gel SDS-PAGE of phage T4 single and dual site display. The virions were CsCl purified and the gel was stained with Coomassie blue. Lanes: 1. T4-SOC/mE2—HOC/E2, arrows show 84 kDa HOC/E2 and 26 kDa SOC/mE2 bands; 2. T4-SOC/mE2, arrow shows 26 kDa SOC/mE2 band; 3. protein markers (from top to bottom: 97, 66, 43, 31, 21 kDa); 4. phage T4 vector T4-Zh⁻; 5. plasmid pSOC-mE2 expression of the strong SOC/mE2 band.

FIG. 13 shows Dual sites displayed phage T4-SOC/mE2-HOC/E2 (CsCl purified): immune capture assay by Western blotting. In FIG. 13A, Rabbit serum against CSFV-E2 was used as first antibody. In FIG. 13B, rabbit serum against T4-HOC as first antibody, Goat IgG anti-rabbit conjugated with HRP was used as second antibody for both FIGS. 13A and B.

FIGS. 14A and B shows EM of two phage T4 strains. Phage particles were negatively stained with gold conjugated SPA. A. phage strain T4-SOC/mE2; B. negative contol vector phage T4-Zh⁻.

FIG. 15 shows ELISA measurement of various type of the T4 phage displayed CSFV immunogens (all samples in this assay were CsCL purified). HE2D: double sites phage T4-Soc/mE2-Hoc/E2; PE2: plasmid expressed Gel-band-cut purified CSFV-E2 protein; SmE2: single site phage T4-S/mE2; HE2/T4: wild type phage T4+D/host BL21 (DE3) infected; BL21(DE3): host control; ZH: phage vector T4-Zh⁻; NR: non-immunized animal serum as contrast.

FIG. 16 shows immunized mice serum antibody level time-course monitor. Legend: B-3-imm:before the tertiary immunization; 3-2W: 2 weeks after tertiary immunization; 3-4W: 4 weeks after tertiary immunization; 3-6W: 6 weeks after tertiary immunization. Other symbols were the same as in Table 3.

FIG. 17 shows ELISA of CSFV cell virion as coat-antigen.

FIG. 18 shows ELISA of CSFV-E2 as coat-antigen.

FIG. 19 shows ELISA of plasmid-SOC/mE2 as coat-protein.

FIG. 20 shows ELISA of wild phage T4⁺D as coat-antigen.

FIG. 21 shows the results of spleen lymphocyte transformation efficiency test of immunized mice with T4-CSFV recombinant phages.

### DETAILED DESCRIPTION OF THE INVENTION

In accordance with the present invention, there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise. The terms “polynucleotide(s)”, “nucleic acid molecule(s)”, “nucleic acid sequences” and “nucleic acids” will be used interchangeably.

Furthermore, the following terms shall have the definitions set out below.

As defined herein “isolated” refers to material removed from its original environment and is thus altered “by the hand of man” from its natural state.

The amino acid residues described herein are preferred to be in the “L” isomeric form. However, residues in the “D” isomeric form can be substituted for any L-amino acid residue, as long as the desired functional is retained by the polypeptide. NH₂ refers to the free amino group present at the amino terminus of a polypeptide. COOH refers to the free carboxy group present at the carboxy terminus of a polypeptide.

“Nucleic acid construct” is defined herein, is a nucleic acid molecule, either single- or double-stranded, which is isolated from a naturally occurring gene or which has been modified to contain segments of nucleic acid which are combined and juxtaposed in a manner which would not otherwise exist in nature. The term nucleic acid construct is synonymous with the term “expression cassette” when the nucleic acid construct contains all the control sequences required for expression of a coding sequence of the present invention.

The term “coding sequence” is defined herein as a portion of a nucleic acid sequence which directly specifies the amino acid sequence of its protein product. The boundaries of the coding sequence are generally determined by a ribosome binding site (prokaryotes) or by the ATG start codon (eukaryotes) of the first open reading frame at the 5′-end of the mRNA and a transcription terminator sequence located just downstream of the open reading frame at the 3′-end of the mRNA. A coding sequence can include, but is not limited to, DNA, cDNA, and recombinant nucleic acid sequences.

A “heterologous” peptide, protein or nucleic acid sequence of a recombinant T4 phage particle is an identifiable segment of a peptide, protein or nucleic acid within a larger peptide, protein or nucleic acid molecule that is not found in association with the larger molecule in nature.

A “vector” may be any vector (e.g., a plasmid or virus) which can be conveniently subjected to recombinant DNA procedures and can bring about the expression or integration of the nucleic acid sequence.

An “origin of replication” refers to those DNA sequences that participate in DNA synthesis.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence. Transcriptional and translational control sequences are DNA regulatory sequences, such as promoters, enhancers, polyadenylation signals, terminators, and the like, that provide for the expression of a coding sequence in a host cell.

A cell has been “transformed” by exogenous or heterologous nucleic acid when such nucleic acid has been introduced inside the cell. The transforming nucleic acid may or may not be integrated (covalently linked) into chromosomal nucleic acid making up the genome of the cell. In prokaryotes, yeast, and mammalian cells for example, the transforming nucleic acid may be maintained on an episomal element such as a plasmid. With respect to eukaryotic cells, a stably transformed cell is one in which the transforming nucleic acid has become integrated into a chromosome so that it is inherited by daughter cells through chromosome replication. This stability is demonstrated by the ability of the eukaryotic cell to establish cell lines or clones comprised of a population of daughter cells containing the transforming nucleic acid.

It should be appreciated that also within the scope of the present invention are nucleic acid sequences encoding the polypeptide(s) of the present invention, which code for a polypeptide having the same amino acid sequence as the sequences disclosed herein, but which are degenerate to the nucleic acids disclosed herein. By “degenerate to” is meant that a different three-letter codon is used to specify a particular amino acid.

The term “peptide” refers to a polymer of at least four amino acids. It may encompass a protein in its entirety or a portion of the protein having functional activity. In a particular embodiment, the peptide has substantially the same activity of the protein. This term also does not refer to or exclude post-expression modifications of the polypeptide, for example, glycosylations, acetylations, phosphorylations and the like. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring.

A nucleic acid molecule is “operatively linked” to an expression control sequence when the expression control sequence controls and regulates the transcription and translation of nucleic acid sequence. The term “operatively linked” includes having an appropriate start signal (e.g., ATG) in front of the nucleic acid sequence to be expressed and maintaining the correct reading frame to permit expression of the nucleic acid sequence under the control of the expression control sequence and production of the desired product encoded by the nucleic acid sequence. If a gene that one desires to insert into a recombinant DNA molecule does not contain an appropriate start signal, such a start signal can be inserted in front of the gene.

As defined herein, a “gene” is the segment of DNA involved in producing a polypeptide chain; it includes regions preceding and following the coding region, as well as intervening sequences (introns) between individual coding segments (exons).

As defined herein, T4 iPII and iPIII are genes encoding T4 internal protein II (IPII) and internal protein III (IPIII) respectively.

As defined herein, an “antigen” is a molecule that stimulates production of antibodies

As defined herein, an “immunogen” is a molecule that stimulates immune response, production of antibody related to both T and B cells.

As defined herein, a “ligand” is a substance that binds to a receptor.

As defined herein, a “HOC” protein is a T4 highly antigenic capsid protein and is encoded by the hoc gene.

As defined herein, a “SOC” protein is a T4 small outer capsid protein and is encoded by the soc gene.

As defined herein, a “receptor” is a molecule that receives or responds to a specific substance.

T4 Expression System

The hoc or soc negative T4 expression system of the present invention may be obtained by homologous recombination of a hoc and/or soc negative phage and a T4 phage particle devoid of T4 lysozyme and/or T4 endonuclease V function, also referred to as a e- and/or denV-T4 phage particle. The expression system may contain variant e and/or denV gene sequences as well as wild type or variant ipII and ipIII gene sequences. These variant sequences may contain substitutions, deletions or insertions resulting in sequences encoding inactive T4 lysozyme and/or T4 endonuclease V.

In the event that the T4 expression system is a hoc negative system, a hoc negative phage is used to obtain said T4 expression system. If the T4 expression system is a soc negative system, a soc negative phage is used. Alternatively, a hoc and soc negative phage may also be used for both hoc and soc T4 expression systems. Such phages may be obtained using procedures known in the art such as by genetic crossing or homologous recombination (see, for example, Homyk and Weil, Virology 61:505-523, Karam, J. D. (ed.), Molecular Biology of Bacteriophage T4. ASM Press, Washington D.C., Black, 1974, Virology 60:166-179; Ren et al., 1997, Gene 195:303-311; Ren et al., 1996, Protein Sci. 5:1833-1843 and Malys et al., 2002, J. Mol. Biol. 319:289-304). The presence or absence of hoc and/or soc gene expression, as noted above, may be determined by PCR using hoc and/or hoc primers or Western Blot analysis using antibodies or antisera to hoc and/or soc (see, for example, Black, 1974, Virology 60:166-179; Ren et al., 1997, Gene 195:303-311; Ren et al., 1996, Protein Sci. 5:1833-1843 and Malys et al., 2002, J. Mol. Biol. 319:289-304).

The T4 phage particle is devoid of T4 lysozyme and/or T4 endonuclease V function and optionally devoid of IPII and IPIII function also referred to as a e⁻ and/or denV⁻, ipII-, ipIII-T4 phage particle may also be obtained using procedures known in the art (see, for example, Black, 1974, Virology 60:166-179, Emrich, 1968, Virology 35:158-165). A particular example of such a particle is Eg326 (S12+, ipI⁻, Alt⁻, e⁻, denV⁻) disclosed in Hong and Black, 1993, Gene 136:193-198.

Recombination procedures are well known in the art and as described in the Examples herein.

The T4 expression system of the present invention may be isolated using methods known in the art. In a particular embodiment, the T4 phage particles may be tested for egg white lysozyme independent and dependent growth. If the resulting phage is e⁻, the phage would only grow in the presence of egg white lysozyme. These particles would be isolated and the presence or absence of expression of soc, hoc would be determined using methods known in the art. These include but are not limited to PCR using ipI, soc or hoc primers or Western Blot analysis using antibodies or antisera to ipI, soc or hoc (see, for example, Black, 1974, Virology 60:166-179; Ren et al., 1997, Gene 195:303-311; Ren et al., 1996, Protein Sci. 5:1833-1843 and Malys et al., 2002, J. Mol. Biol. 319:289-304). The resulting particles containing the recombinant T4 expression system of the present invention may be further isolated using procedures known in the art such as sucrose gradient centrifugation, CsCl gradient centrifguation, glycerol centrifugation (see, for example, Mooney et al., 1987, J. Virol. 61:2828-2834, Ren et al., 1996, Protein Sci. 5:1833-1843).

In the event that the T4 expression system is both soc and hoc negative, the following procedure may be used. Specifically, a hoc⁻ and soc⁻ recombinant T4 phage expression system may undergo homologous recombination using procedures described above and in the examples set forth as well as using procedures known in the art.

SOC/HOC Fusion Proteins

The invention is further directed to SOC and HOC fusion peptides or proteins. SOC or HOC sequences are bound to heterologous peptides or proteins. As noted above, these peptides or proteins may be a receptor, ligand, pathogen, antigen, immunogen antibody or bio-drugs. In a particular embodiment, the peptide or protein is an FMDV peptide or protein and may encompass the VPI, VPII, VPIII and/or VPIV proteins.

The SOC protein has the amino acid sequence (Macdonald et al., 1984, Genetics 106:17-27): mastrgyvni ktfeqkldgn kkiegkeisv afplysdvhk isgahyqtfp sekaaystvy eenqrtewia anedlwkvtg (SEQ ID NO:4)

The HOC protein has the following amino acid sequence (Kaliman et al., 1995, Nucl. Acids Res. 18 (14), 4277): mdikvhfhdf shvridcees tfhelrdffs feadgyrfnp rfrygnwdgr irlldynrll pfglvgqikk fcdnfgykaw idpqinekee lsrkdfdewl skleiysgnk riephwyqkd avfeglvnrr rilnlptsa (SEQ ID NO:5)

The SOC or HOC peptide may contain conservative amino acid substitutions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain. Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter the specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, as well as these in reverse. Alternatively, the nucleotide sequence encoding PDX-1 may contain alterations which produce silent substitutions, additions, or deletions, but do not alter the properties or activities of the encoded polypeptide. Nucleotide variants produced by silent substitutions due to the degeneracy of the genetic code are preferred.

The fusion protein or peptide may be obtained using recombinant DNA methods. For example, a nucleic acid sequence encoding a heterologous peptide may be inserted into a vector containing nucleic acid sequences encoding the HOC or HOC peptide or SOC or SOC peptide (for example, see FIGS. 4A and 4B). In a particular embodiment, the HOC or SOC sequence is obtained by PCR and inserted, for example, into a pET vector, or other protein expression vector capable of expressing a fusion protein. In a more particular embodiment, the nucleic acid sequence encoding HOC is shown below in SEQ ID NO:6:

| | |
|---|---|
| 1 | ATGACTTTTA CAGTTGATAT AACTCCTAAA ACACCTACAG GGGTTATTGA |
| 51 | TGAAACTAAG CAGTTTACTG CTACACCCAG TGGTCAAACT GGAGGCGGAA |
| 101 | CTATTACATA TGCTTGGAGC GTAGATAATG TTCCACAAGA TGGAGCTGAA |
| 151 | GCAACTTTTA GTTATGTACT AAAAGGACCT GCCGGTCAAA AGACTATTAA |
| 201 | AGTAGTTGCA ACAAATACAC TTTCTGAAGG AGGCCCGGAA ACGGCTGAAG |
| 251 | CGACAACAAC TATCACAGTT AAAAATAAGA CACAGACGAC TACCTTAGCC |
| 301 | GTAACTCCTG CTAGTCCTGC GGCTGGAGTG ATTGGAACCC CAGTTCAATT |
| 351 | TACTGCTGCC TTAGCTTCTC AACCTGATGG AGCATCTGCT ACGTATCAGT |
| 401 | GGTATGTAGA TGATTCACAA GTTGGTGGAG AAACTAACTC TACATTTAGC |
| 451 | TATACTCCAA CTACAAGTGG AGTAAAAAGA ATTAAATGCG TAGCCCAAGT |
| 501 | AACCGCGACA GATTATGATG CACTAAGCGT TACTTCTAAT GAAGTATCAT |
| 551 | TAACGGTTAA TAAGAAGACA ATGAATCCAC AGGTTACATT GACTCCTCCT |
| 601 | TCTATTAATG TTCAGCAAGA TGCTTCGGCT ACATTTACGG CTAATGTTAC |
| 651 | GGGTGCTCCA GAAGAAGCAC AAATTACTTA CTCATGGAAG AAAGATTCTT |
| 701 | CTCCTGTAGA AGGGTCAACT AACGTATATA CTGTCGATAC CTCATCTGTT |
| 751 | GGAAGTCAAA CTATTGAAGT TACTGCAACT GTTACTGCTG CAGATTATAA |
| 801 | CCCTGTAACC GTTACCAAAA CTGGTAATGT AACAGTCACG GCTAAAGTTG |
| 851 | CTCCAGAACC AGAAGGTGAA TTACCTTATG TTCATCCTCT TCCACACCGT |
| 901 | AGCTCAGCTT ACATCTGGTG CGGTTGGTGG GTTATGGATG AAATCCAAAA |
| 951 | AATGACCGAA GAAGGTAAAG ATTGGAAAAC TGACGACCCA GATAGTAAAT |
| 1001 | ATTACCTGCA TCGTTACACT CTCCAGAAGA TGATGAAAGA CTATCCAGAA |
| 1051 | GTTGATGTCC AAGAATCGCG TAATGGATAC ATCATTCATA AAACTGCTTT |
| 1101 | AGAAACTGGT ATCATCTATA CCTATCCATA ATCATAAGGG GCTTCGGCCC |
| 1151 | CTTTCTTCAT TTTGAAAGCA CACAAAACAC AATCAGAAAA TGATGTATAT |
| 1201 | AATGGCACCA ACTCGATAAC ATGA |

In one embodiment, the nucleic acid sequence encoding a heterologous peptide is immediately adjacent to the sequence encoding HOC or SOC or HOC or SOC functional peptide. In another embodiment, the two sequences are separated by a linker or spacer sequence. In a specific embodiment, the linker or spacer sequence may comprise a cleavage site (e.g., trypsin cleavage site).

The fusion protein may be expressed in a eukaryotic or prokaryotic cell. In a preferred embodiment, the fusion protein would be expressed in a prokaryotic cell (e.g., E. coli) and would be isolated and purified using procedures known in the art, such as HPLC and column chromatography.

Recombinant T4 Phage Particles

The recombinant T4 phage particles of the present invention contain or express a SOC and/or HOC fusion peptide. In a particular embodiment, the T4 phage particle is iPII and iPIII negative as well as e and/or denV negative, also referred to as ipII⁻, ipIII⁻, e⁻ and/or denV⁻. The recombinant T4 phage particle of the present invention may be obtained by first transforming the prokaryotic cell, preferably E. coli with a vector comprising a nucleic acid sequence encoding the hoc or soc fusion protein. If the prokaryotic cell is transformed with a HOC fusion plasmid, the transformed cell is subsequently infected with a hoc⁻ recombinant expression system of the present invention. If the prokaryotic cell is transformed with a SOC fusion plasmid, the transformed cell is subsequently infected with a soc⁻ recombinant expression system of the present invention. If the prokaryotic cell is transformed with a HOC and SOC fusion plasmid, the transformed cell is subsequently infected with a hoc⁻soc⁻ recombinant expression system of the present invention.

Plaques are isolated and checked for integration of desired heterologous nucleic acid sequences using methods known in the art, e.g., PCR primers.

Vaccines

The recombinant T4 phage particles of the present invention may be used to formulate compositions. In a particular embodiment, the compositions of the present invention may be immunogenic or antigenic compositions and may thus be used in vaccine formulations. In a particular embodiment, the compositions and particularly the vaccine formulations of the present invention may comprise recombinant T4 phage particles expressing one or more HOC- and/or SOC-: FMDV, HIV, porcine circovirus, AIV, vvIBDV, HBV, encephalitis and/or measles virus fusion peptides. The compositions, particularly, the vaccine formulations may also comprise recombinant T4 phage particles of the present invention expressing HOC and/or SOC-: EGF, EFGR, VEGF and/or VEGFR fusion peptides.

In a more particular embodiment, the compositions may be multivalent vaccines. For example, one composition may comprise FMDV peptides from two or more serotypes; another composition may comprise HIV and tuberculosis peptides or a multiple HIV peptides; another composition may comprise antigenic or immunogenic peptides from two or more avian infectious diseases, such as chicken very virulent infectious bursal disease virus (vvIBDV) and NDV (chicken Newcastle Disease Virus). Another composition may comprise antigens from bacteria infecting fish. For example, Streptococcus iniae and Streptococcus agalactiae has been found to cause diseases found in 22 species of cultured and wild fish such as tilapia and rainbow trout; Flavobacterium columnare causes columnaris disease in many varieties of fish; Edwardsiella tarda; Vibrio anguillarum, biotype I and II, V. harveyi, V. parahaemolyticus and V. vulnificus affects shrimp.

In a more particular embodiment, the composition of the present invention may comprise the recombinant T4 phage particles of the present invention that express a HOC- and/or SOC- The vaccine delivery systems of the present invention can be prepared in a physiologically acceptable formulation, such as in a pharmaceutically acceptable carrier, using known techniques. For example, the recombinant T4 phage particles of the present invention may be combined with a pharmaceutically acceptable excipient to form an immunogenic composition. Alternatively, these recombinant T4 phage particles may be administered in a vehicle having specificity for a target site, such as a tumor or infection.

The vaccine delivery vehicles of the present invention may be administered in the form of a solid, liquid or aerosol. Examples of solid compositions include but are not limited to pills, creams, and implantable dosage units. Pills may be administered orally. Therapeutic creams may be administered topically. Implantable dosage units may be administered locally, for example, at a tumor site, or may be implanted for systematic release of the therapeutic composition, for example, subcutaneously. Examples of liquid compositions include but are not limited to formulations adapted for injection intramuscularly, subcutaneously, intravenously, intra-arterially, and formulations for topical and intraocular administration. Examples of aerosol formulations include inhaler formulations for administration to the lungs.

The compositions may be administered by standard routes of administration. In general, the composition may be administered by topical, oral, rectal, nasal or parenteral (for example, intravenous, subcutaneous, or intramuscular) routes. In addition, the composition may be incorporated into sustained release matrices such as biodegradable polymers, the polymers being implanted in the vicinity of where delivery is desired, for example, at the site of a tumor. The method includes administration of a single dose, administration of repeated doses at predetermined time intervals, and sustained administration for a predetermined period of time.

A sustained release matrix, as used herein, is a matrix made of materials, usually polymers which are degradable by enzymatic or acid/base hydrolysis or by dissolution. Once inserted into the body, the matrix is acted upon by enzymes and body fluids. The sustained release matrix desirably is chosen by biocompatible materials such as liposomes, polylactides (polylactide acid), polyglycolide (polymer of glycolic acid), polylactide co-glycolide (copolymers of lactic acid and glycolic acid), polyanhydrides, poly (ortho) esters, polypeptides, hyaluronic acid, collagen, chondroitin sulfate, carboxylic acids, fatty acids, phospholipids, polysaccharides, nucleic acids, polyamino acids, amino acids such phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

The dosage of the vaccine composition made according to the present invention depends on the species, breed, age, size, vaccination history, and health status of the animal to be vaccinated. Other factors like antigen concentration, additional vaccine components, and route of administration (i.e., subcutaneous, intradermal, oral, intramuscular or intravenous administration) will also impact the effective dosage. The dosage of vaccine to administer is easily determinable based on the antigen concentration of the vaccine, the route of administration, and the age and condition of the animal to be vaccinated. Each batch of antigen may be individually calibrated. Alternatively, trials of different dosages, as well as LD₅₀ studies and other screening procedures can be used to determine effective dosage for the immunogenic or antigenic composition of the present invention. From the examples presented below, it will be readily apparent what approximate dosage and what approximate volume would be appropriate for using the vaccine composition described herein. The critical factor is that the dosage provides at least a partial protective effect against natural infection, as evidenced by a reduction in the mortality and morbidity associated with natural infection. The appropriate volume is likewise easily ascertained by one of ordinary skill in the art. For example, in avian species the volume of a dose may be from about 0.1 ml to about 0.5 ml and, advantageously, from about 0.3 ml to about 0.5 ml. For feline, canine and equine species, the volume of a dose may be from about 0.2 ml to about 3.0 ml, advantageously from about 0.3 ml to about 2.0 ml, and more advantageously, from about 0.5 ml to about 1.0 ml. For bovine and porcine species, the volume of dose may be from about 0.2 ml to about 5.0 ml, advantageously from about 0.3 ml to about 3.0 ml, and more advantageously from 0.5 ml to about 2.0 ml.

Repeated vaccinations may be preferable at periodic time intervals to enhance the immune response initially or when a long period of time has elapsed since the last dose. In one embodiment of the present invention, the vaccine composition is administered as a parenteral injection (i.e., subcutaneously, intradermally, or intramuscularly. The composition may be administered as one dose or, in alternate embodiments, administered in repeated doses of from about two to about five doses given at intervals of about two to about six weeks, preferably from about two to about five weeks. However, one of skill in the art will recognize that the number of doses and the time interval between vaccinations depends on a number of factors including, but not limited to, the age of the animal vaccinated; the condition of the animal; the route of immunization; amount of antigen available per dose; and the like. For initial vaccination, the period will generally be longer than a week and preferably will be between about two to about five weeks. For previously vaccinated animals, a booster vaccination, before or during pregnancy, at about an annual interval may be performed.

Patients may be humans or animals. For example, T4 phage particles expressing FMDV peptides would be useful in treating pigs and cows; T4 phage particles containing pig circle virus. T4 phage particles expressing avian virus proteins may be useful in treating birds, which also includes chicken and duck.

The methods and compositions described herein are useful for treating human and animal diseases and processes including but not limited to bacterial disease, fungal disease, rickettsial disease, chlamydial disease, viral disease parasitic infection, sexually transmitted diseases, sarcoidosis, and prion disease. The methods and compositions described herein are also useful for treating any disease or disorder mandating an immune response.

Pesticide Made by T4 Expression System

The bacterium, Bacillus thuringiensis (Bt), has proven to be a rich source of toxins for killing insect pests. Most of the toxin genes now being used in genetic engineering are produced in Genetic modified food (GM) crops. These genes are designated Cry family (up to 41 Cry genes), Cyt family. The Cry family gene toxins target specific insect cell receptor proteins and create membrane-pores that lead to kill the insect gut cells. Only a few Cry genes have found favor in genetically modified (GM) crops. Cyt genes have been characterized that are genetically distinct from Cry genes and target on the insect blood cells. Other alternatives, vegetative insecticidal proteins (VIP), have been found to have potent broad spectrum activity against insects, too. VIP genes are not homologous to Cry and Cyt genes and bind to cell membrane proteins different from the other toxins.

Diagnostic Methods and Kits

The particles of the present invention may be used for diagnostic purposes. In a specific embodiment, the expressed HOC or SOC fusion peptides are linked to detectable label. In a most specific embodiment, the detectable label is a fluorescent or luminescent protein such as green fluorescent protein, golden, or yellow fluorescent protein. The kit may comprise a library of the recombinant T4 phage particles of the present invention. These libraries comprise a multiplicity of the T4 phage particles of the present invention. These phage particles may express nucleic acid sequences encoding HOC and/or SOC-heterologous antigenic and immunogenic fusion peptides as well as antibodies.

In one embodiment, the kit of the present invention may contain a library of recombinant T4 phage particles expressing FMDV peptides of various serotypes. These peptides would be capable of binding to antibodies to FMDV since they are antigenic or immunogenic. The binding of any of the peptides with a patient sample would indicate FMDV infection. Either the T4 phage particles and/or patient sample may be labeled with a detectable label.

In another embodiment, the kits of the present invention may comprise a library of recombinant T4 phage particles expressing HOC and/or SOC-avian influenza virus (AIV) antigenic or immunogenic fusion peptides. In a particular embodiment, these peptides would be derived from the AIV HA protein. The kit may contain recombinant T4 phage particles expressing HOC and/or SOC-AIV peptides of various serotypes. Furthermore, the kit of the present invention may contain recombinant T4 phage particles expressing HOC and/or SOC AIV, Marek's disease virus (MDV), infectious bursal disease virus (IBDV), infectious laryngotracheitis virus (ILTV), avian encephalomyelitis (AEV), chick anemia virus (CAV), Fowipox virus (FPV), avian influenza virus (AIV), avian reovirus, avian leukosis virus (ALV), reticuloendotheliosis virus (REV), avian adenovirus and/or hemorrhagic enteritis virus (HEV) antigenic or immunogenic fusion peptides.

In yet another embodiment, the kit may comprise T4 phage particles expressing HOC and/or SOC canine distemper virus, feline distemper virus, canine parvovirus, canine adenovirus antigenic or immunogenic fusion peptides.

In yet another embodiment, the kit may comprise recombinant T4 phage particles expressing HOC and/or SOC mycobacterium (tuberculosis) antigenic or immunogenic peptides including but not limited to purified protein derivative (PPD), Bacillus of Calmette and Guerin (BCG) antigen and/or A60-antigen antigenic or immunogenic peptides. The kit may comprise peptides from a plurality of mycobacterium strains. The kit may be of particular use in determining if there is cross contamination, e.g., cow's milk and human sample.

The kit in a further embodiment the kit may comprise recombinant T4 phage particles that may express HOC and/or SOC antigenic and/or immunogenic fusion peptides derived from swine infertility and respiratory syndrome (SIRS) virus, porcine circovirus, PRRSV (e.g., from the envelope glycoprotein), porcine classic swine fever virus (CSFV) (e.g., E2, mE2).

In yet another embodiment, the kit may comprise recombinant T4 phage particles that may express antigen and/or immunogenic peptides derived from human HBV (e.g., HBsAg, HBcAg or HBeAg), encephalitis (VP1 protein) and/or measles (e.g., HA).

In a further embodiment the kit of the present invention may comprise recombinant T4 phage particles that may express antigen and/or immunogenic peptides derived from human dengue fever virus.

In one embodiment, an antibody engineering library may be constructed, specifically a scFV or dcFV (single chain or double chain antibody variation domain, especially for monoantibody gene library) gene is cloned into the T4 expression system of the present invention and scFV or dcFV peptide is expressed on the T4 phage surface. In a preferred embodiment, pathogen is detected directly in with higher sensitivity and accuracy.

High Throughput Screening (FACS 3-D platform), Biopanning (Solid Phase 2-D Platform)

The invention is further directed to methods and kits for identifying ligands binding to particular receptors or particularly, high throughput screening of possible drug or vaccine candidates. In a particular embodiment, a library of the recombinant T4 phage particles of the present invention are contacted with a target substance, such as an antigen or immunogen of interest. In a particular embodiment, the target substance may be a protein receptor. This receptor may actually be expressed on a recombinant T4 phage particle as a SOC and/or HOC fusion peptide or Phage Expression, packaging, processing system (PEPP) (Biotechniques 1998, 25:1008-1012) and may also contain a detectable label, such as a fluorescent protein. In a particular embodiment, the recombinant T4 phage particles and/or antigen or immunogen are labeled with a detectable label. The presence or absence of binding of the recombinant phage particles to a target substance is subsequently detected using methods known in the art, e.g., FACS 3-D platform or by a solid phase 2-D platform.

In another particular embodiment, a recombinant T4 phage particle containing a receptor and label expressed as a HOC or SOC fusion protein is contacted with a peptide library. The presence or absence of binding of peptides in the library to said recombinant T4 phage particle may be detected using methods known in the art.

### EXAMPLES

### Example 1

### High Efficiency T4 Bacteriophage Surface Protein Expression System: Vectors and Construction

The construction of three systems are described:

1.) ΦμTΔSoc+p IN-Soc (T4 phage Soc site expression system)
2.) ΦT4ΔHoc+p IN-Hoc (T4 phage Hoc site expression system)
3.) ΦT4ΔSoc ΔHoc+p IN-Soc-Hoc (T4 phage Soc-Hoc bipartite sites expression system):

Recombination between ΦT4ΔSoc and ΦT4ΔHoc to create the double deletion mutant without damaging the other genes functions.

ΦT4ΔSoc+p IN-Soc (T4 Phage Soc Site Expression System)

The following procedure was used in obtaining the above T4 phage Soc site expression system This expression system is more efficient than previously published systems and contains several unique endonuclease sites (SmaI, XbaI, SalI, NcoI etc.) at the EcoRI site to facilitate heterologous gene insertion;

The T4 phage, T4-A9.8 Soc was crossed with T4 phage eG326 in one host E. coli CR63 using the homologous recombination procedure described above. T4-Δ9.8Soc was described in Homyk et al., 1974, Virology 61:505-523. This phage T4-Δ9.8 Soc contains approximately a 9.8 kb deletion between T4 genes 39 and 56. T4 phage strain eG326 was described in Hong Y R, Black L W. 1993, Gene 136:193-198.

E. coli CR63 in CM medium (per liter contains: 10 g Tryptone, 5 g NaCl, 50 ml Tris.CL, pH 7.5, 10 ml 25% Na-Citrate), at 0.2 (OD600), was inoculated with both phage T4-Δ9.8Soc and eG326 at MOI (multiplicity of infection) 0.5 as described in Ren et al., 1996, Protein Science 5: 1833-1843. The culture was incubated at 35° C. overnight, spread on lysozyme 200 ug/ml containing and no lysozyme CM plates. The plates were incubated at 37° C. overnight. The PFU number on the lysozyme plate was double the amount than on the lysozyme deficient plate. All PFUs from the lysozyme containing plate were collected, inoculated into E. coli CR63 again and the process was repeated. The PFU number was much larger in lysozyme containing plates than on lysozyme deficient plates. Phage particles from second round lysozyme plate were collected and the process was repeated a third time. 20 single plaques were selected at random from the lysozyme plate and subjected to PCR analysis for Soc gene deletion and IPII-IPIII deletion.

Anti-Soc antibody was used as a probe to screen the 9.4 kDa Soc protein band deletion by Western Blotting. All plaques found to contain the SOC deletion by PCR analysis was also found to be SOC negative via Western Blot analysis. No Soc positive protein bands appeared. Some were IPII-IPIII negative; others were not.

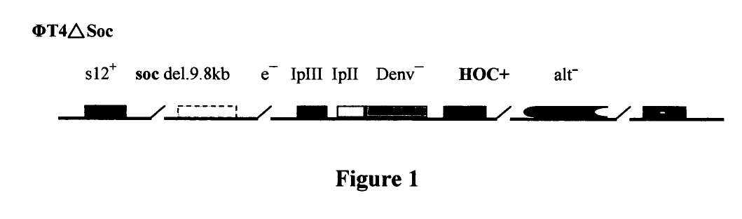

The final vector is shown in FIG. 1.

ΦT4ΔHoc+p IN-Hoc (T4 Phage Hoc Site Expression System)

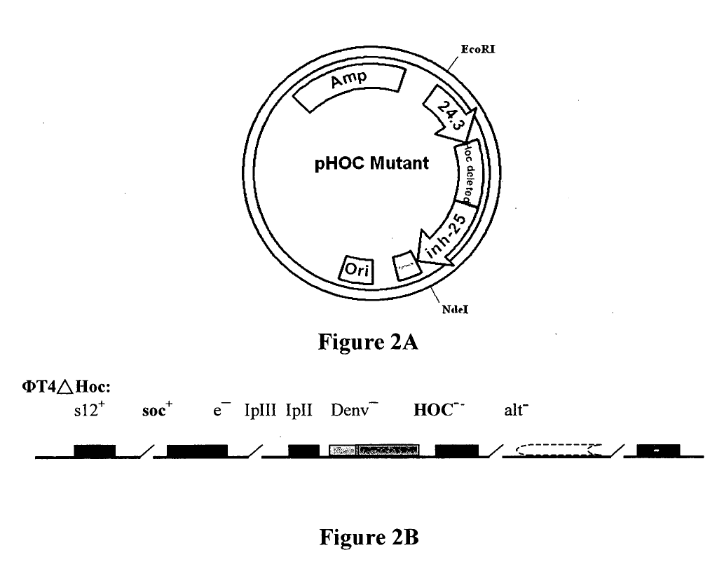

First, PΔHoc376aa is obtained and is shown in FIG. 2A. This contains approximately a 376 aa (1128 bp) deletion between genes 24 and inh-25. PΔHoc376aa was constructed as follows. The Soc-V3 fragment was removed from the plasmid vector, pE V-3 (see Ren et al., 1996, Protein Science 5:1833-1843) using restriction enzymes NdeI and EcoRI as vector. The fragment to be inserted into pE was obtained by ligating two PCR fragments together using wild T4 phage DNA as template. For the first PCR, one primer was located at far hoc gene end, the 186 bp position, the opposite primer extended around ˜500 bp away in T4 24 gene area. For the second PCR, one primer was located at far hoc gene end, at 156 bp position; the opposite primer extended around ˜500 bp away in T4 inh-25 gene area. These two PCR pieces were ligated together as ˜1 kb insert into pE and transformed into E. coli. CR63. A homologous recombination reaction was undertaken by infecting transformed E. coli. CR63 with wild T4 phage. The conditions were analogous to those described above for T4 phage soc site expression system. E. coli CR63 was grown in CM medium, while OD600 to around 0.2, inoculated with T4 wild phage at MOI 0.5, and cultured at 35° C. overnight. Plaques were spread on common LB plates and grown at 37° C. overnight. All plaques were collected and the process was repeated two more times. Isolated plaques were screened for the Hoc deletion via PCR and Western Blot analysis using an anti-Hoc antibody.

The final vector is shown in FIG. 2B.

ΦT4ΔSoc-ΔHoc+p IN-Soc-Hoc (T4 Phage Soc-Hoc Bipartite Sites Expression System)

The T4 phage Soc-Hoc bipartite sites expression vector was obtained by homologous recombination between ΦT4ΔSoc and ΦT4ΔHoc in E. coli CR63. The first step involved a homologous recombination reaction between ΦT4ΔSoc and ΦT4ΔHoc. 9.

OD 600 E. coli CR63 was infected with T4 phage mutants ΦT4ΔSoc and ΦT4ΔHoc together and incubated as described above. E. coli CR63 was grown in CM medium (OD600 to around 0.2), inoculated with phage ΦT4ΔSoc and ΦT4ΔHoc both at MOI 0.5, and cultured at 35° C. overnight, spread on common LB plates, and grown at 37° C. overnight. All plaques were collected, and the process was repeated twice.

20 resulting phage particles were collected and screened for Soc and Hoc deletions by PCR and by Western Blotting using anti-Hoc and anti-Soc antibodies.

A couple of CM plates were prepared. One was lysozyme containing and another was lysozyme free. A plaque containing Soc and Hoc deletions, and lysozyme dependence was isolated.

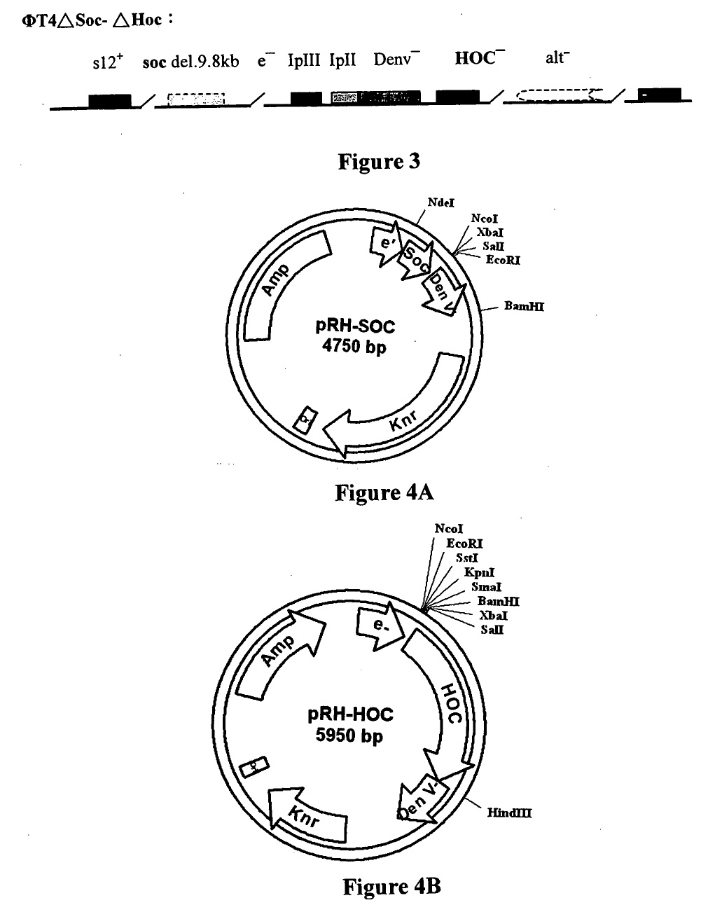

The final vector is shown in FIG. 3.

### Example 2

### Application of T4 Phage Surface Expression System to FMDV Vaccines Including T4 Phage FMDV P1 Vaccine and T4 Phage FMDV Subunit Vaccine

RNA was extracted from FMDV O-serotype with ZOIL (Promega). Two primers were designed for PCR reactions of FMDV O-serotype P1:

| | |
|---|---|
| 5′ CTCAACGCAGAATGGAAAGCA 3′ | (SEQ ID NO:1) |
| 5′ GGTCGAAGTTCAGAAGCTGTT 3′ | (SEQ ID NO:2) |

RT-PCR reagents were used to amplify the FMDV P1 O-serotype gene with SEQ ID NO: 1 and SEQ ID NO:2.

PCR product amplified FMDV P1 gene was cloned into T4 phage expression vector pRH-Soc (FIG. 4A) to obtain pRH-Soc-P1. The P1 (˜2250 bp) PCR product was inserted into Vector T-easy of Promega Inc. PCR cloning Kit; a white colony was selected and plasmid DNA was extracted. The PCR product was cut with EcoRI, inserted into pRH-Soc (=pRH) cut by enzyme EcoRI. The correct direction recombinant clone was selected by PCR. DNA homologous recombination was carried out between pRH-Soc-P1 and phage T4-Soc deleted vector using similar procedures to those described in Example 1. The resulting recombinant T4 particle was verified by PCR and FMDV antibody immuno-test.

E. coli CR63 was subsequently infected with the T4 particle and cultured in LB medium to express phage-T4 Soc-FDMV-P1 viral particle for vaccine purposes. In a large flask, E. coli CR63 in 5000 ml EM or LB medium, was grown to OD550 0.1, infected with phage-T4 Soc-FDMV-P1 at MOI>1 at 30-35° C. and grown until phage lysate appeared and can be seen visibly (around 8-9 hours); the lysate period time was influenced by culture temperature. The same infection can be repeated several times, until visual lysate is more strong. The phage was harvested through a—centrifugation process with 6000 RPM-20000 RPM to remove the host cell debris.

Both Western blotting and dot-ELISA assay were used to test the phage T4-Soc-FMDV-P1 antigen with anti-FMDV serums from Pig, Cow, Goat. The results were positive.

The phage-T4 Soc-FDMV-P1 vaccine has been tested with mice and pigs.

Results from two studies are shown below. Before strong virus challenge, mice were immunized mice with T4 particles listed below 2 times, within two weeks intervals. The infectious dose (TCID50) of virus was determined in adult animal. 100,000 TCID50 FMD virus mix with 1:50 diluted immunized adult animal's sera was inoculate. The effect was determined 7 days post infection.

FMDV challenge test

| | Adult | Young | | | |
|---|---|---|---|---|---|
| | mice | mice | | | |
| (group) | number | number | Death | Live | Protection |
| Study #1 | | | | | |
| T4-FMDV-P1 | 10 | 30 | 0 | 30 | 100% |
| T4 FMDV-P1 oral | | | | | |
| T4-FMDV-P1 | 10 | 30 | 0 | 30 | 100% |
| T4-FMDV-P1 Injection | | | | | |
| T4-FMDV-F3 | 5 | 15 | 7 | 8 | 53% |
| T4-FMDV-F3 oral | | | | | |
| T4-FMDV-F3 | 5 | 15 | 4 | 11 | 73% |
| T4-FMDV-F3 injection | | | | | |
| Negative Control | 5 | 15 | 15 | 0 | 0% |
| T4-FMDV-P1 | | | | | |

| | | | |
|---|---|---|---|
| T4-FMDV-P1 injection | 2 Pigs | 0 | 2 |

| | Young mice | | | |
|---|---|---|---|---|
| Group | number | Death | Live | Protection |
| Study #2 | | | | |
| | 6 | 0 | 6 | 100% |
| T4-FMDV-P1 oral | 9 | 0 | 9 | 100% |
| T4-FMDV-P1 injection | 5 | 0 | 5 | 100% |
| T4-FMDV-F3 oral | 12 | 4 | 8 | 66% |
| T4-FMDV-F3 injection | 12 | 3 | 9 | 75% |
| T4 phage particle alone | 7 | 7 | 0 | 0% |
| negative control | 5 | 15 | 0 | 0% |

T4-FMDV-P1 T4 FMDV O-type P1 vaccine
T4-FMDV-F3 T4 phage O-type international O1K type subunit vaccine F3 containing an FMDV-antigen epitope arrangement: 56 aa length, on VP1, aa141-160-200-213-141-160 plus linkers.

### Example 3

### Bacteriophage T4 Capsid Surface SOC and HOC Bipartite Display with Enhanced Classical Swine Fever Virus Immunogenicity

Materials and Methods

CSFV Antigen Expression Plasmid, T4 Phage Display System

Plasmids pcDSW and pETmE2 for eukaryotic cell expression of whole length CSFV-E2 and for prokaryotic cell expression of CSFV mE2 respectively were constructed as previously described (Xing et al, 2001, Vaccine 19:1520-1525). The codons in pETmE2 have been mutagenized to E. coli coding bias codons. The T4 phage display system included phage display vector T4-Zh⁻ and plasmid vectors pE-SII, pRH, and pTHOC. Phage vector T4-Zh⁻ was generated by recombination between T4 phage Z-1 (soc gene deleted) (Ren et al. 1996, Protein Science 5: 1833-1843.) and T4 phage amhoc (hoc gene amber mutation) (Ren et al., 1997, Gene, 195: 303-311). The desired phage recombinant, identified by lysozyme dependent growth and absence of SOC and HOC proteins, was used in these studies. The T4 related plasmids were previously described by Ren et al (Ren et al. 1996, Protein Science 5: 1833-1843; Ren et al., 1997, Gene 195: 303-311; Ren and Black, 1998, Gene 215:439˜444). Host bacteria E. coli 594 (sup^{o}), CR63 (sup D), DH5a, BL21(DE3), M109(DE3), HMS174(DE3), and wild-type T4D phage, were used.

Expression Recombination Plasmid Construction of CSFV Antigen Fused with T4-soc and T4-hoc Gene: pSmE2. pSE2. pHOCE2

First, the CSFV mE2 gene was fused with the soc gene by PCR with the following primers:
Pr.1: 5′GAATCATATGGCTAGTACTCGCGGT3′ (bold letters: NdeI site) (SEQ ID NO:7)
Pr.2: 5′caggctaagcgCAAGCAACCAGTTACTTTCCACAAATC3′ (SEQ ID NO:8)
Pr.3: 5′aactggttgcttgCGCTTAGCCTGCAAGGAAGATTACC3′ (SEQ ID NO:9)
Pr.4: 5′CGGAATTCAAATTGGGCAGACAAGGTAG3′(bold letters: EcoRI site) (SEQ ID NO:10)

The capitalized letters are soc sequences and the lower case letters are CSFV sequences, and the underlined letters show the complementary sequences for annealing. Pr. 1 & Pr2 were matched with pE-SII as template and Pr.3 & Pr.4 were matched with pETmE2 as template. These two resulting PCR products were mixed in equimolar amounts as template together with Pr. 1 and Pr.4 for a second PCR. The resulting fragment was cloned into pE-SII to produce the soc-mE2 gene fused expression vector pSmE2.

The E2 gene was fused with soc by ligation of the three fragments: pE-SII/NdeI cut with EcoRI; pSmE2/NdeI cut with SacI; and pcDSW/SacI cut with EcoRI produced SOC-E2 gene fusion expression plasmid pSE2.

E2 gene was fused with hoc by PCR and pcDSW as template. Primers were:

Pr.5:

(SEQ ID NO:11)

5′ACCATGGTACGCTTAGCCTGCAAGGAAGATTAC3′
(bold letters: NcoI site)
Pr.6:

(SEQ ID NO:12)

5′CGGAATTCGTCAAACCAGTACTGATACTCGCC3′
(bold letters: EcoRI site)

The resulting whole length E2 PCR fragment was inserted into pTHOC/NcoI-EcoRI cut vector to produce the IPTG inducible expression vector pHOCE2 for phage T4 HOC site display. In this section all PCR syntheses were: 98° C. for 5 min; 20 cycles of 95° C. for 50S, 55° C. for 30S, 72° C. for 30S; and ended with 72° C. for 5 min.
Construction of Phage T4 SOC-CSFV Single Site Display Recombinant T4-SOC/mE2. T4-SOC/E2 and Dual Sites Display Recombinant T4-SOC/mE2—HOC/E2

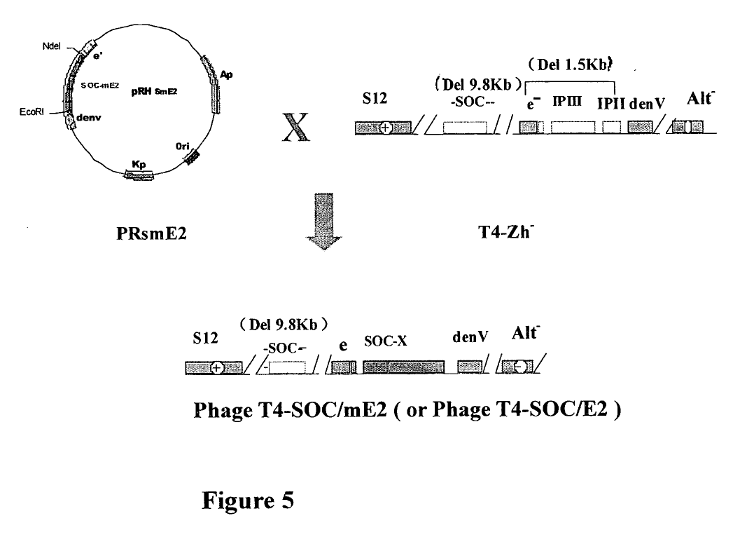

For phage T4 SOC-single site display: plasmid pSmE2 and pSE2 were digested with NdeI and EcoRI, the inserts mE2 and E2 were isolated, and then cloned into phage T4 integration vector pRH, to yield pRsmE2 and pRsE2. They were transformed into E. coli CR63 (sup D) and infected with phage T4-Zh⁻. All procedures for selecting target antigen CSFV mE2 and E2 fragment integrated T4 phages were by the same lysozyme—independent growth method described in Ren and Black, 1996, Protein Science. 5: 1833-1843); recombinant phages T4-SOC/mE2, T4-SOC/E2 were produced. The strategy and phage recombinants are shown in FIG. 5.

The following procedure was used for phage T4 SOC-, HOC-dual sites display: E. coli BL21(DE3) harboring pHOCE2 was grown to OD₆₀₀ 0.1 in MC medium (Ren and Black, 1996, Protein Science 5: 1833-1843) containing ampicillin 50 ug/ml, and induced with 1 mM IPTG, 28° C., grown to OD₆₀₀ 0.3-0.4, infected with phage T4-SOC/mE2 at a multiplicity of infection (M.O.I.) of 0.2, grown for an additional 4-5 hours. A few drops of chloroform were then added and mixed, and the cell debris removed by centrifugation at 4000×g for 5 min. The resulting phage, T4-SOC/mE2-HOC/E2, in supernatant was put into a dialysis tube (MW 8000-14400,WDSS). This dialysis tube was imbedded into solid PEG8000 powder in a beaker and held overnight. This led to the phage being concentrated to 10⁸ pfu/ml and containing pHOCE2 protein around 10 μg/ml (see Table 3), to produce partially purified phage, named non-purified type. In order to compare dual sites and single site antigen display immunological effects, all strains of phage T4 displays CSFV immunogen (T4-SOC/mE2, T4-SOC/E2 and T4-SOC/mE2—HOC/E2) particles were further purified by CsCl density gradient centrifugation (Ren and Black, 1996, Protein Science 5: 1833-1843), and used in the immunity tests. All such phage T4 display samples including single and double site displayed that underwent CsCl step gradient density centrifugation were named purified type phages.

Immunization of Mice

100 female BALB/3C mice, weight 18-20 grams, were randomly divided into 7 groups. The detailed administration and immunization dose of all groups is shown in Table 3.

TABLE 3
100 mice immunization arrangement profile with various type of T4
phage displayed CSFV antigens

| | | | | D.SmE2-HE2, | | | |
|---|---|---|---|---|---|---|---|
| groups | S.SmE2,P | S.SE2,P | D.SmE2-HE2, P | N.P. | gel-cut-E2 | T4-Zh,P | blank |
| Mouse | 15 | 15 | 15 | 15 | 15 | 15 | 10 |
| number | | | | | | | |
| Dose of each | 10¹⁰ pfu | 10¹⁰ pfu | 10¹⁰ pfu | 10 μg | 20 μg | 10¹⁰ pfu | Not |
| mouse | | | | 10⁸ pfu | | | immunized |

Legend:
S.SmE2,P T4 phage single site SOC-mE2 displayed, CsCl purified;
S.SE2,P T4 phage single site SOC-E2 displayed, purified;
D.SmE2-HE2,P T4 phage dual sites SOC/mE2-HOC/E2 displayed, purified;
D.SmE2-HE2, N.P. T4 phage dual sites SOC/mE2-HOC-E2 displayed, non-purified;
gel-cut-E2 gel-cut-purified E2 protein as positive control;
T4-Zh,P phage vector T4-Zh CsCl purified as negative background;
blank non-immunized mice as negative control. The amount of plasmid expressed antigen protein was measured by spectrophotometer using BCA protein kit (Pierce Inc. USA).

Groups of 15 mice were each immunized with 1×10¹⁰ pfu of single site or dual sites displaying T4 phage samples. Six experimental groups were immunized three times at two week intervals. Each mouse was injected subcutaneously on the dorsal hind quarters. The first immunization was performed with Freund's Complete Adjuvant containing 1 mg/ml Mycobacterium tuberculosis H37Ra (Sigma). The second immunization was with Incomplete Freund's Adjuvant, the third immunization with no adjuvant. Each mouse was bled via the tail vein on the first day prior to immunization and after tertiary immunization at 2, 4, and 6 weeks. All mice in all groups were sacrificed at 6 weeks after tertiary immunization and each mouse spleen was resected for lymphocyte transformation efficiency testing.

Immunity Detection of Phage T4 Single Site and Dual Site Displayed CSFV Antigens

Antibodies:

Mouse sera immunized with mE2 (MS), rabbit sera immunized with mE2 (RS), rabbit sera immunized with full length E2 (RFS), mouse sera immunized with CSFV-E2 DNA vaccine (MDNA)(28), mE2 monoclonal antibody prepared with CSFV-E2 DNA vaccine (Ma et al., Chin J. Vet Sci, 22:121-124), pig sera immunized with CSFV vaccine (PS), and rabbit serum against T4 HOC protein were used.

Coating Antigens:

1. CSFV cell virions were prepared as follows: 2×50 ml of PK15 cell (American Type Culture Collection:ATCC-33) were grown to monolayers in MEM, washed two times with antibiotics-free and serum-free MEM. Then 5 ml of 2% blood CSFV was inoculated, kept at 37° C. for 2 hours, washed three times, then MEM containing 2% horse serum was added and cells were cultured at 37° C. for 72 hours. The top 45 ml medium was poured off, and 5 ml residual medium was kept together with the bottom cells, which were frozen at −20° C. and thawed three times with mixing, and ultrasonicated to reduce viscosity. Cell debris was removed from the antigens extract by centrifugation at 5000 rpm for 5 min.

2. SDS-PAGE gel-band-cut purified E. coli BL21(DE3) expressed whole length CSFV—E2 protein;

3. E. coli BL21(DE3) expressed SOC-mE2 inclusion body was dissolved in 8M urea.

ELISA

1. The following assay was configured for detecting antigenicity of phages T4 single and dual site displayed immunogens with different CSFV antibodies. The ELISA steps were conducted as described in Engvall et al., 1971, Immunochemistry 8:871-879: The 96 wells tray was coated overnight at 4° C. with 10⁹⁻¹⁰ pfu of display phage T4 per well, or 20 ug of gel purified blocked at 37° C. with 200 ul of 3% gelatin for 1 hour, then washed four times with 0.05% Tween-20 PBS, each wash for three minutes. Mouse antiserum against CSFV-mE2 was diluted 1:500 with pH7.4 PBS solution containing 2% BSA, 10% E. coli lysate and 10% wild-type T4+D phage lysate. The diluted serum was added to each well, the tray was shaken at 37° C. for 1 hour, washed and goat anti mouse IgG conjugated with HRP enzyme was added and shaken for 30 min. After washing, the wells were colored by adding OPD(o-phenylenediamine). and the OD₄₉₀, was measured and analyzed with SPSS 8.0 (ISBN) statistical software.

2. This assay was designed for measuring the serum antibody levels in all groups of phage T4 samples immunized mice with different coating antigens. The tray wells were coated with plasmid pSOC-mE2 expressed antigen (1 ug/well). Every mouse serum specimen was diluted 1:100, otherwise the assay procedure was the same as the above ELISA. In addition, in order to further confirm the specificity of the elicited mouse serum antibody, the following test was performed: To measure the antibody specificity of unpurified double sites phage T4-SOC/mE2-HOC/E2 only, each tray wells was coated with wild-type T4D+phage in the amount of 10⁹ pfu, while a parallel positive control with pSOC-mE2 expressed protein antigen (1 ug/well) was set up to measure the serum specimens at 4 weeks after tertiary immunization.

Mice Spleen Lymphocyte Transformation Efficiency Test

All immunized mice spleens were resected out under sterile conditions, and to each spleen 10 ml RPMI1640 medium (GIBCO Inc) was added, then homogenized and passed through a 200 millimicron copper mesh-boult. The spleen cells were collected and centrifuged at 1500 rpm for two min, the pellets were washed two times with PRM1640 medium, then suspended to 2×10⁷ cells per ml.

On 96 well polystyrene ELISA trays, 100 ul of the above spleen cell suspension was first added to each well, then in different assays the following antigen stimulators were added: 1. mitogen conA to 2.5 ug/ml; 2. gel-band-cut purified E. coli expressed CSFV-E2 protein to 10 ug/ml; 3. CSFV cell virons to 1 ug/ml; 4. same volume of RPMI 1640 as inside-group blank control. Each sample test was repeated in 3 wells. The trays were cultured in 5% CO₂, 37° C. for 68 hours, then 10 ul of 5 mg/ml MTT (Sigma) was added. Culture growth continued for 4 hours, and 100 ul DMSO was added incubated in the dark for 15 minutes. The OD₅₇₀ absorbance was measured with an ELISA microplate reader (Bio-Rad, ELX 800, USA). The resulting data were statistically treated with the SPSS 8.0 (ISBN) software as above.

Results

CSFV Antigens from Protein Expression Plasmids

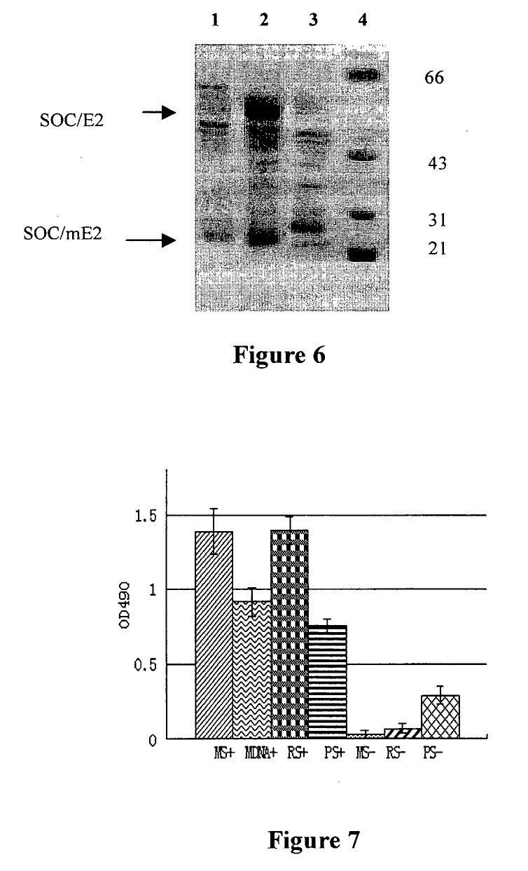

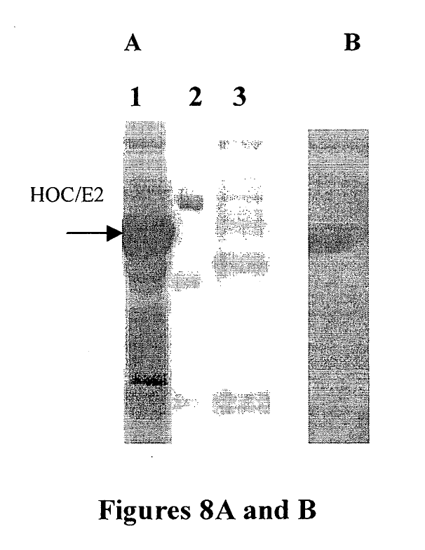

The expression plasmids pSmE2 and pSE2 were constructed to express CSFV proteins fused to the carboxy-terminus of the phage T4 SOC protein. When the plasmid DNAs were sequenced, in pSmE2 the soc-fused mE2 insert had a length of 621 bp, whereas in pSE2 the soc-fused E2 had a length of 1365 bp and both CSFV coding sequences were correct and in the correct soc reading frame. Following transformation into BL21(DE3) and induction with IPTG, pSmE2 expressed a 26 kDa fusion protein, and pSE2 expressed a 53 kDa fusion protein (FIG. 6). Because in protein CSFV E2 most significant antigenic domains are located on the mE2 fragment, the immunological tests were mainly conducted with mE2. Western blotting with CSFV pig antiserum verified a distinct 26 kDa positive pSmE2 band upon SDS-PAGE. The antigenicity of plasmid expressed SOC-mE2 fused protein was determined by ELISA employing all of the antibodies listed in FIG. 7 (For antisera, see Materials and Methods). The results show that SOC-fused mE2 specifically bound with all the positive CSFV antisera but not with control non-immune sera from the same animals. Plasmid pHOCE2 was constructed to express protein CSFV E2 fused to the aminoterminus of the phage T4 HOC protein. PCR amplified DNA employing primers Pr.5 and Pr.6 and plasmid pHOCE2 as template showed that the insert had the desired length. IPTG induced plasmid pHOC-E2 expressed a 84 kDa protein fusion band, and Western blotting with pig anti-CSFV antiserum showed this protein was reactive (FIG. 8).

CSFV Immunogens Displayed on the Bacteriophage T4 Capsid Surface

Display on the SOC Site:

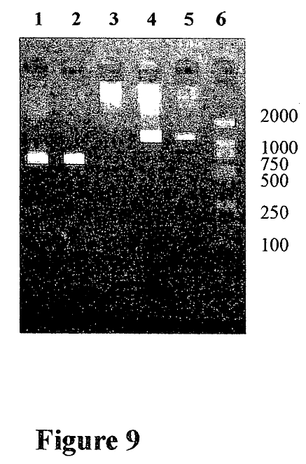

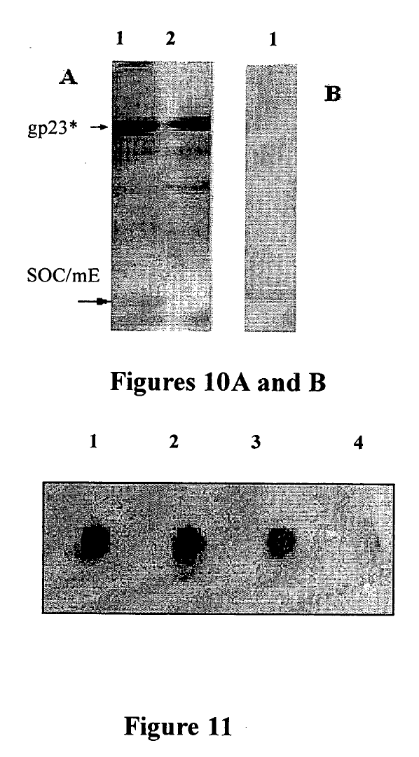

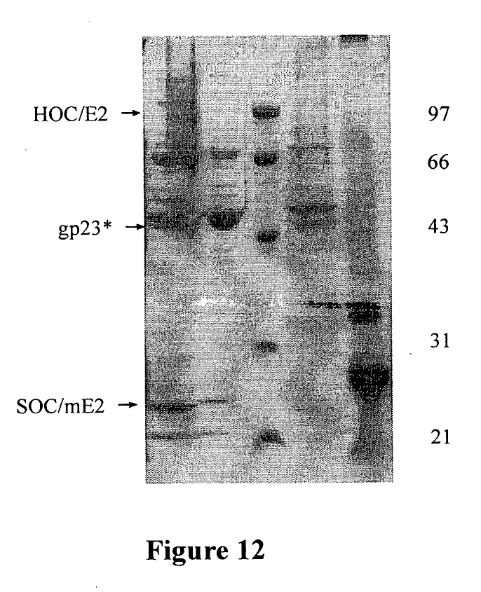

Through plasmid—phage homologous recombination of plasmids pRsmE2 (e′-SOC/mE2—denV) and pRsE2 (e′-SOC/E2—denV) with phage vector T4-Zh, the desired recombinant T4 phage strains T4-SOC/mE2 and T4-SOC/E2⁻ were obtained respectively. Phage genomic DNA was extracted as template and when matched with the primer pairs Pr. 1-Pr.4 and Pr. 1-Pr.5, PCR exhibited the 621 bp mE2 and the 1365 bp E2, indicating that CSFV antigen coding target fragments were integrated into the T4 phage genome (FIG. 9). The recombinant phage proteins were denatured and run on a 12% SDS-PAGE gel and a 26 kDa SOC-mE2 band was seen. Western Blotting with pig CSFV-antiserum showed a corresponding mE2 positive band (FIG. 10). Scanning with a UV thin-layer scanner (CS-9301PC Shimadzu JEPEN) showed that the mE2 band constituted 5.052% of the total T4 phage proteins (FIG. 10, FIG. 12); thus the amount of displayed SOC-mE2 was estimated to be from 170 to 280 molecules per virion. By the same analysis, phage T4 surface displayed SOC-E2 was estimated at around 50-90 copies, whereas HOC-E2 was expressed at around 30-60 copies per virion.

Display on Both the SOC and HOC Sites:

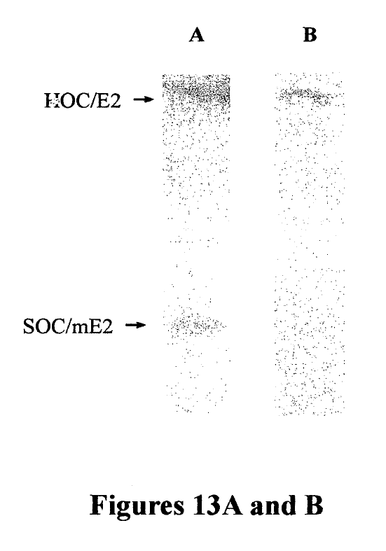

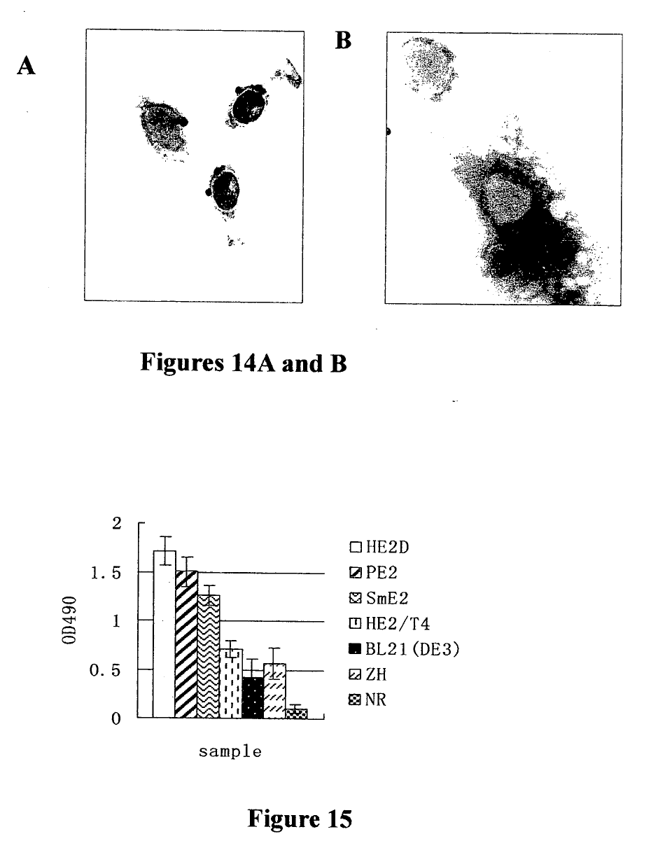

Dot-ELISA with T4-SOC/mE2—HOC/E2 and T4-SOC/mE2 showed clearly positive spots (FIG. 11). On a 12% SDS-PAGE gel, dual SOC plus HOC sites display resulted in two target antigen bands: 26 kDa SOC/mE2 and 84 kDa HOC/E2. However, single site display only yielded one 26 kDa SOC/mE2 antigen band upon Coomassie blue staining (FIG. 12). Western blotting with rabbit anti CSFV-E2 antibody detected both SOC/mE2 (26 kDa) and HOC/E2 (84 kDa) positive bands, whereas rabbit anti HOC protein antibody only showed the HOC/E2 (84 kDa) single positive band (FIG. 13). The results demonstrate that the HOC fused E2 antigen protein was successfully displayed from the HOC site of phage T4-SOC/mE2 which is constructed by the T4 phage bipartite display vector T4-Zh⁻.

Electron Micrograph of Phage T4-SOC/mE2 Particle

The CsCl purified recombinant phage T4-SOC/mE2 virions and the blank display phage vector T4-Zh⁻ virons were negatively stained with gold—labeled SPA (Sigma). As shown in FIG. 10, it can be observed that the gold particles specifically adhered to T4-SOC/mE2 particles but not T4-Zh⁻ particles, suggesting that the SOC-mE2 antigen is displayed from the T4 phage surface of the recombinant.

Comparison of Phage T4 Single and Double Site Displayed CSFV-E2 Antigenicity

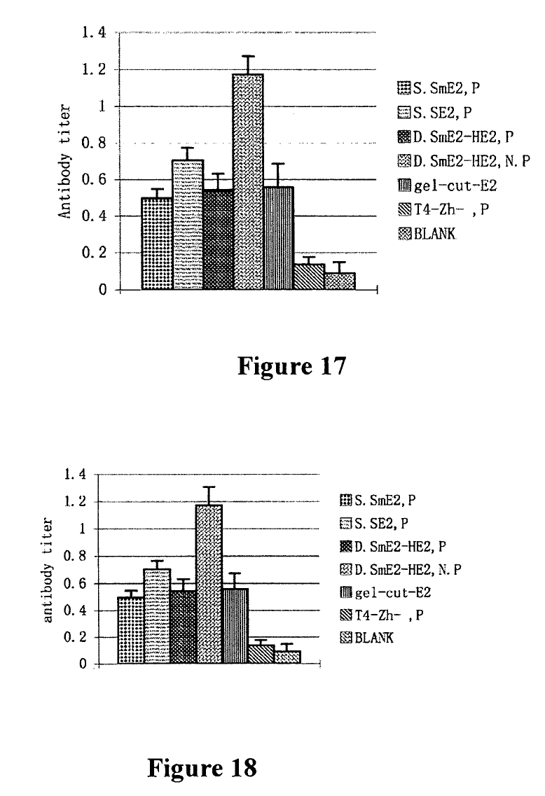

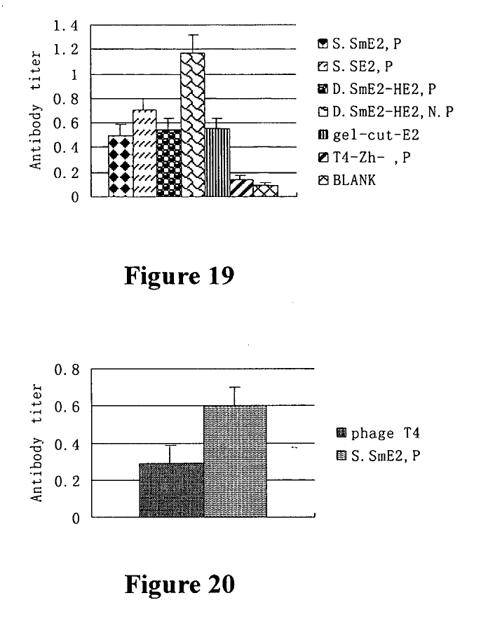

ELISA data demonstrated that compared with blank vector T4-Zh⁻, as well as wild-type phage T4 and E. coli host bacteria, the CSFV antigenicity of CsCl purified dual site display phage T4 was higher than plasmid expressed protein (FIG. 15). In the case of non-CsCl purified phage, dual site display phage showed much higher antigenicity than plasmid expression in immunity tests (FIGS. 17, 18, 19).

Study of the Humoral Immunity of Phage T4 Single and Dual Site Displayed CSFV Recombinants

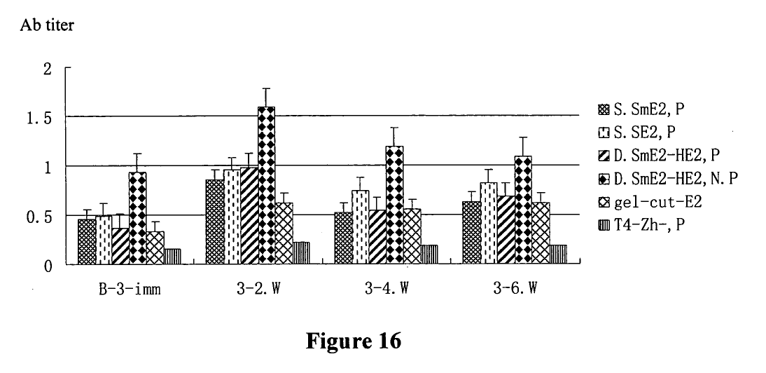

100 mice were injected subcutaneously with the following phage T4 preparations: single site antigen T4-SOC/mE2, T4-SOC/E2, and blank phage T4-Zh⁻ (as control); all were CsCl purified. As to double site antigen display, strain T4-SOC/mE2—HOC/E2 was injected in one group as non-purified phage and in another group as CsCl purified phage respectively (see Table 3). The time course of mice antibody levels in vivo was monitored and is plotted in FIG. 16, which indicates that except for the negative control and blank groups, in all five groups the serum specific CSFV antibodies were clearly produced upon secondary immunization. After tertiary immunization, the serum antibody levels increased. Later on, the antibody levels slowly fell, but comparatively high CSFV antibody titer levels were maintained for more than 56 days in serum. Interestingly, among all 7 groups, the dual sites SOC/mE2-HOC-E2 unpurified antigen group induced extremely high antibody levels that were significantly higher than the other groups (P<0.01). After tertiary immunization, even the CSFV antibody titer of phage T4 single site displayed SOC/E2 groups was obviously higher than the plasmid expressed E2 group (P<0.05).

The Effects of using Different Antigens on the ELISA Detection of Mice Sera Antibody Levels

Three different CSFV antigens were coated onto ELISA trays (as shown in Materials and Methods) and antibody titers of mice sera specimens at 4 weeks post-tertiary immunization was determined. The resulting data demonstrated that all five groups of immunized mice responded immunologically to three different CSFV antigens including native CSFV cell virions, and plasmid expressed mE2 and E2 (FIGS. 17, 18 and 19). As a control, blank vector T4-Zh gave a lower OD value (P<0.05). The results show that the recombinant phage T4 induced CSFV specific antibodies in mice. To further verify the specificity, wild-type T4+D phage and phage T4-SOC/mE2, both containing the SOC antigen component, were used in parallel on coated ELISA trays to determine the antibody levels in mice of the non-purified T4-SOC/mE2—HOC/E2 group at 4 weeks post-tertiary immunization. The data show the OD₄₉₀ value of T4-SOC/mE2 is much higher than T4+D (P<0.01) (FIG. 20). Overall the data suggest that the recombinant phage T4 induced antibodies against both SOC and T4 skeleton protein antibodies, but CSFV specific antibodies were dominant.

The Cellular-Immunity Study of Phage T4 Dual Site Displayed Recombinants with CSFV

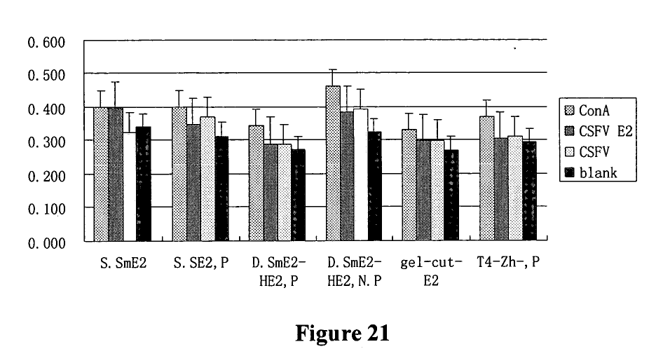

Spleen lymphocytes of all 100 mice (including 10 mice of the blank control group) (see Table 3) at 6 weeks post-tertiary immunization were used for lymphocyte switching rate determination. The resulting data are presented in FIG. 21. In all six immunized mice groups the non-antigen-stimulated data was set as an inside-group blank control. The mitosis accelerant ConA was used as non-specific stimulator. In all three stimulator antigen status groups, the spleen lymphocyte response to ConA in all 6 immunized groups was obviously stronger than the blank control (P<0.05). In the case of plasmid expressed CSFV-E2 as stimulator antigen, the lymphocyte switching rate of all groups was only a little higher than the CSFV cell virion stimulator antigen, and the rate of all immunized groups was higher than the inside-group control. It is worth noting that in phage T4-SOC/E2 group and T4-SOC/mE2—HOC/E2 (unpurified) group the rate was significantly higher than the control (P<0.05). The lymphocyte response to CSFV whole virion in all 6 immunized groups was better than to CSFV-E2 antigen. The lymphocyte switching rate tests demonstrate that mice specifically respond immunologically to phage T4 recombinants.

Discussion

This study ascertained that CSFV proteins E2 and mE2 could be expressed as fusion proteins to the phage T4 SOC and HOC proteins. When expressed as fusion proteins from either the phage genome (SOC fusions), or expression plasmids (HOC fusions), the fusion proteins were bound to the SOC and HOC sites of T4 phage capsids lacking these two dispensable capsid surface proteins. Phage containing these fusion proteins were highly immunogenic. All single and dual site display T4 phage, as well as plasmid expressed E2, showed that the CSFV proteins reacted with mouse, rabbit, and pig CSFV related antisera and monoclonal antibodies. In vivo, the murine immune response tests in this study involved both humoral and cellular immunity, and showed that the recombinant T4 display phage nanoparticles administered to mice elicited high titers of CSFV antigen specific antibodies. The SOC and HOC surface displaying phage induced serum-antibodies that can recognize not only plasmid expressed mE2 and E2 proteins, but also entire CSFV virions (FIGS. 17, 18, 19). To substantiate the role of CSFV—related T4 capsid proteins in animal CSFV antibody induction, a test was conducted in which wild type T4 virions were used as control in comparison with T4 SOC/mE2 fusion phage. As shown in FIG. 20, the antibody titer was distinctly different (p<0.01), strongly suggesting that it is the phage T4 displayed CSFV antigens that are specifically immunogenic.

It is noteworthy that under the experimental conditions employed in the experiment using partially purified phage samples in an in vivo immune-response test, the T4 phage particle amount with dual antigen sites (10⁸ level) was only 1% of single antigen site (10¹⁰ level, see Table 3), but induced equal or higher antibody levels in mice. If immunized with unpurified dual antigen sites displaying T4 phage, containing plasmid expressed HOC-E2 soluble antigen (around 100 ug/ml in mixed phage suspension), an even more efficient animal immune-response was obtained.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Various references are cited herein, the disclosures of which are incorporated by reference in their entireties.

What is claimed is:

1. A recombinant T4 phage particle comprising a SOC and/or HOC fusion peptide, wherein said phage particle has inactive T4 endonuclease V and/or T4 lysozyme function.

2. The recombinant T4 phage particle according to claim 1, wherein said fusion peptide comprises SOC and/or HOC bound to a heterologous peptide selected from the group consisting of a ligand, receptor, toxin, antigen or immunogen.

3. The recombinant T4 phage particle according to claim 1, wherein said fusion peptide comprises SOC and/or HOC bound to a tumor antigen or receptor peptide selected from the group consisting of epidermal growth factor (EGF), epidermal growth factor receptor (EGFR), vascular endothelial growth factor (VEGF) and vascular endothelial growth factor receptor (VEGFR).

4. The recombinant T4 phage particle according to claim 2, wherein said heterologous peptide is a toxin, antigen or immunogen derived from a pathogen.

5. The recombinant T4 phage particle according to claim 4, wherein said heterologous peptide is derived from a pathogen selected from the group consisting of Human Immunodeficiency Virus (HIV), Foot and Mouth Disease Virus (FMDV), poliovirus, avian encephalomyelitis (AEV), Avian Influenza Virus (AIV), avian adenovirus, avian leukosis virus (ALV), avian reovirus, Fowlpox virus (FPV), human Dengue Fever Virus, infectious bursal disease virus (IBDV), Marek's disease virus (MDV), hepatitis B virus, human measles virus, and classical swine fever virus (CSFV), canine distemper virus, feline distemper virus, canine adenovirus, mycobacterium, Streptococcus iniae, Streptocococcus agalactiae, Flavobacterium columnare, Edwardsiella tarda, Vibrio anguillarum biotype I and II, V. Harveyi, V. parahaemolyticus V. vulnificus and Bacillus thuringiensis.

6. The recombinant T4 phage particle according to claim 1, wherein said particle comprises one or more of the heterologous peptides derived from CSFV.

7. The recombinant T4 phage particle according to claim 1, wherein said particle comprises a HOC- and/or SOC bound to a heterologous peptide selected from the group consisting of CFSV E2 and mE2 peptides.

8. The recombinant T4 phage particle of claim 2, wherein said heterologous peptide is one or more HIV peptides selected from the group consisting of p24, gp41, tat and/or vpr peptides.

9. The recombinant T4 phage particle of claim 1, wherein said particle expresses one or more HOC or SOC-FMDV fusion peptides.

10. The recombinant T4 phage particle according to claim 1, wherein said particle expresses a HOC or SOC FMDV VP1, VP2, VP3 and/or VP4 fusion peptide.

11. The recombinant T4 phage particle according to claim 1, wherein said particle expresses a HOC and SOC fusion peptide.

12. A hoc⁻ and/or soc⁻, ipII⁺ and/or ipIII⁺ T4 phage expression system wherein said expression system is additionally e⁻, and/or denV⁻.

13. A method for obtaining a hoc⁻ or soc⁻ T4 phage expression system of claim 12 comprising
(a) providing a soc⁻ or hoc⁻ T4 phage;
(b) providing a soc⁻ and hoc⁻, e⁻ and/or denV⁻ T4 phage particle, wherein (a) and (b) are both soc⁻ or both hoc⁻;
(c) culturing a prokaryotic cell with (a) and (b) under conditions promoting recombination of (a) and (b) and
(d) isolating said expression system.

14. The method according to claim 13, wherein said soc or hoc T4 phage particle is obtained by deleting nucleic acid sequences encoding SOC or HOC proteins or portions thereof.

15. A method for obtaining the hoc and soc negative T4 phage recombinant expression system of claim 12 comprising
(a) providing a hoc negative recombinant T4 phage expression system;
(b) providing a soc negative recombinant T4 phage expression system;
(c) culturing (a) and (b) in a prokaryotic cell under conditions promoting recombination of (a) and (b) and
(d) isolating said expression system.

16. A method for obtaining a HOC and/or SOC fusion peptide comprising
(a) providing a vector comprising a nucleic acid sequence encoding a HOC or SOC fusion peptide;
(b) providing the recombinant T4 expression system of claim 12;
(c) culturing a prokaryotic cell with the expression vector of (a) and expression system of (b) to obtain said T4 phage expression system under conditions to obtain recombination of (a) and (b) and expression of said heterologous peptide and
(d) isolating said peptide.

17. A method for obtaining the hoc and/or soc negative recombinant T4 particle of claim 1 comprising
(a) providing a vector comprising a nucleic acid sequence encoding a SOC and/or HOC fusion peptide;
(b) providing a recombinant hoc⁻ and/or soc⁻ expression system of claim 12;
(c) culturing prokaryotic cell with the expression vector of (a) and expression system of (b) to under conditions to obtain recombination of (a) and (b) and obtain said recombinant T4 particle and
(d) isolating said recombinant T4 particle.

18. An isolated fusion peptide comprising HOC or SOC bound to a heterologous peptide derived from a pathogen selected from the group consisting of Human Immunodeficiency Virus (HIV), Foot and Mouth Disease Virus (FMDV), poliovirus, avian encephalomyelitis (AEV), Avian Influenza Virus (AIV), avian adenovirus, avian leukosis virus (ALV), avian reovirus, Fowlpox virus (FPV), human Dengue Fever Virus, infectious bursal disease virus (IBDV), Marek's disease virus (MDV), hepatitis B virus, human measles virus, and classical swine fever virus (CSFV), mycobacterium, Streptococcus iniae, Streptocococcus agalactiae, Flavobacterium columnare, Edwardsiella tarda, Vibrio anguillarum biotype I and II, V. Harveyi, V. parahaemolyticus V. vulnificus, and Bacillus thuringiensis.

19. An isolated nucleic acid molecule encoding the fusion peptide of claim 18.

20. A construct comprising the isolated nucleic acid molecule of claim 19.

21. A prokaryotic cell comprising the isolated nucleic acid molecule of claim 19.

22. A prokaryotic cell comprising the recombinant T4 phage particle of claim 1.

23. A prokaryotic cell comprising the recombinant T4 phage expression system of claim 12.

24. An isolated nucleic acid molecule encoding SOC and/or HOC fusion peptide, T4 IPII and/or T4 IPIII or functional peptides thereof, but is devoid of sequences encoding functional T4 lysozyme and T4 endonuclease V.

25. A nucleic acid construct comprising the isolated nucleic acid molecule of claim 24.

25. A vector comprising the nucleic acid molecule of claim 24.

26. A prokaryotic cell comprising the nucleic acid molecule of claim 24.

27. A method for obtaining a HOC and/or SOC fusion protein comprising
(a) culturing the host prokaryotic cell of claim 21 and
(b) isolating the HOC and/or SOC protein.

28. A method for obtaining a HOC and/or SOC fusion protein comprising
(a) culturing the host prokaryotic cell of claim 22 and
(b) isolating the HOC and/or SOC protein.

29. A method for obtaining a HOC and/or SOC fusion protein comprising
(c) culturing the host prokaryotic cell of claim 23 and
(d) isolating the HOC and/or SOC protein.

30. A method for obtaining a HOC and/or SOC fusion protein comprising
(a) culturing the host prokaryotic cell of claim 26 and
(b)isolating the HOC and/or SOC protein.

31. A method for obtaining a HOC and/or SOC fusion protein comprising
(a) culturing the host prokaryotic cell of claim 26 and
(b) isolating the HOC and/or SOC protein.

32. A composition comprising one or more recombinant T4 phage particles of claim 1 and a carrier.

33. The composition according to claim 30 wherein said composition is an antigenic or immunogenic composition.

34. A method of modulating growth of a pathogen in an animal comprising administering to said animal in need thereof the composition of claim 30 in an amount effective to modulate growth of a pathogen in said animal.

35. A method of modulating growth of a pathogen in an animal comprising administering to said animal in need thereof the T4 phage particle of claim 4 in an amount effective to modulate growth of a pathogen in said animal.

36. The recombinant T4 phage particle of claim 4 wherein said heterologous peptide is derived from Bacillus thuringiensis.

35. A composition comprising one ore more recombinant T4 phage particles of claim 36 and a carrier.

36. A method of a method for modulating pathogen infestation in a plant in need thereof comprising applying to said plant an amount of the recombinant T4 phage particle of claim 36 effective to modulate pathogen infestation.

37. A library of recombinant T4 phage particles of claim 1.

38. The library of claim 37, wherein said T4 particle comprises (a) heterologous peptide selected from the group consisting of a ligand, antibody, antigen and immunogen and (b) a detectable label.

39. A method of detecting the presence or absence of a pathogen in a sample comprising incubating the library of claim 1 with said sample and detecting the presence or absence of binding of a phage particle in said library to said sample.

40. A kit comprising the library of claim 37.

41. A method for identifying ligands that bind to receptors comprising
(a) providing a library of peptides;
(b) providing a receptor, wherein said receptor is expressed on the recombinant T4 phage particle of claim 1;
(c) incubating (a) and (b)
(d) detecting binding of (a) and (b).

42. The method according to claim 35, wherein the library of peptides in step (a) is the library of claim 37.
